SEQUENCE LISTING (22 sequences)

SEQ ID NO: 1 - DNA, 12 bp, Artificial Sequence
  Description of Artificial Sequence DNA coding signal peptide
  CDS (1)..(12)
aaggacgagttg

SEQ ID NO: 2 - protein, 4 aa, Artificial Sequence
  Description of Artificial Sequence signal peptide
KDEL

SEQ ID NO: 3 - DNA, 35 bp, Artificial Sequence
  Description of Artificial Sequence forward primer CTB-F-NcoI
atccatggttaaattcaaatttggtgtttttttta

SEQ ID NO: 4 - DNA, 43 bp, Artificial Sequence
  Description of Artificial Sequence reverse primer CTB-KDEL-R
atgagctcttacaactcgtccttatttgccatactaattgcgg

SEQ ID NO: 5 - DNA, 315 bp, Artificial Sequence
  Description of Artificial Sequence mutated gene coding cholera toxinsubunit B
  CDS (1)..(315)
atggcaacacctcaaaatattactgatttgtgtgcagaataccacaacacacagatccacaccctcaatgataagattttctcttatacagaatctctagctggaaagagagagatggctatcattactttcaagaatggtgcaactttccaagtagaagtaccaggcagtcaacatatagattcacaaaagaaggcaattgaaaggatgaaggataccctgaggattgcatatcttactgaagctaaagttgaaaagctatgtgtatggaataataagactcctcatgcaattgccgcaattagtatggcaaat

SEQ ID NO: 6 - protein, 105 aa, Vibrio cholerae
MATPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMAN

SEQ ID NO: 7 - DNA, 78 bp, Artificial Sequence
  Description of Artificial Sequence fragment F1-up of mutated gene coding cholera toxin subunit B
aatttgtgtgcagaataccacaacacacagatccacaccctcaatgataagattttctcttatacagaatctctagct

SEQ ID NO: 8 - DNA, 70 bp, Artificial Sequence
  Description of Artificial Sequence fragment F1-low of mutated gene coding cholera toxin subunit B
agagattctgtataagagaaaatcttatcattgagggtgtggatctgtgtgttgtggtattctgcacaca

SEQ ID NO: 9 - DNA, 92 bp, Artificial Sequence
  Description of Artificial Sequence fragment F2-up of mutated gene coding cholera toxin subunit B
aattgaaaggatgaaggataccctgaggattgcatatcttactgaagctaaagttgaaaagctatgtgtatggaataataagactcctcatg

SEQ ID NO: 10 - DNA, 84 bp, Artificial Sequence
  Description of Artificial Sequence fragment F2-low of mutated gene coding cholera toxin subunit B
aggagtcttattattccatacacatagcttttcaactttagcttcagtaagatatgcaatcctcagggtatccttcatcctttc

SEQ ID NO: 11 - DNA, 46 bp, Artificial Sequence
  Description of Artificial Sequence forward primer F1-Fd
atgcgaattccatggcaacacctcaaaatattactgatttgtgtgc

SEQ ID NO: 12 - DNA, 79 bp, Artificial Sequence
  Description of Artificial Sequence reverse primer F1-Rv
tctacttggaaagttgcaccattcttgaaagtaatgatagccatctctctctttccagctagagattctgtataagaga

SEQ ID NO: 13 - DNA, 68 bp, Artificial Sequence
  Description of Artificial Sequence forward primer F2-Fd
caactttccaagtagaagtaccaggcagtcaacatatagattcacaaaagaaggcaattgaaaggatg

SEQ ID NO: 14 - DNA, 48 bp, Artificial Sequence
  Description of Artificial Sequence reverse primer F2-Rv
cgggatccatttgccatactaattgctgcaattgcatgaggagtctta

SEQ ID NO: 15 - DNA, 78 bp, Oryza sativa
  CDS (1)..(78)
atggcgagttccgttttctctcggttttctatatacttttgtgttcttctattatgccacggttctatggcccagccc

SEQ ID NO: 16 - protein, 26 aa, Oryza sativa
MASSVFSRFSIYFCVLLLCHGSMAQP

SEQ ID NO: 17 - DNA, 2337 bp, Oryza sativa
tctagacagattcttgctaccaacaacttcacaaagtagtagtcaaccaaaactatgctaaggaatcacctcacttccgcccatgaccgtgagcacgactgttcaaacagtttgttaatctctacaaagaaggtacactttacctacacaacgccactaacctgagttacccagcccatgcaaaatagccacgtcttgtgacttaagggatttcgcgacaaggcatttcgaaagcccacacaaggacaccttatgaaaactggaggggtcccacagaccaacaacaagttaggtcccaaaccatgttgtgccaggaaaaatccaaggggtcctccccaacaccaccccgacaaatccacttgtccattggcatcaagatttgcctgacctagctaattactcagccaggcatgtcacaattcacccatgtggtcacacatgttatggttggatgaaattctaaaggaatcggtccatatgagcaagaccgagaaaccataccaccagtacttctaccgaaatacgagtttagtaaactcatttgttttcaaggcacccgacccaggtgtgtcgggttttccagggattttgtaaacccaagttttacccatagttgatcattcaaattttgaggagggtcattggtatccgtacctgagggcacgaatactgagacctagcattgtagtcgaccaaggaggttaatgcagcaattgtaggtggggcctgttggttatattgcaaactgcggccaacatttcatgtgtaatttagagatgtgcattttgagaaatgaaatacttagtttcaaattatgggctcaaaataatcaaaggtgacctaccttgcttgatatcttgagcttcttcctcgtattccgcgcactaggactcttctggctccgaagctacacgtggaacgagataactcaacaaaacgaccaaggaaaagctcgtattagtgagtactaagtgtgccactgaatagatctcgatttttgaggaattttagaagttgaacagagtcaatcgaacagacagttgaagagatatggattttctaagattaattgattctctgtataaagaaaaaaagtattattgaattaaatggaaaaagaaaaaggaaaaaggggatggcttctgctttttgggctgaaggcggcgtgtggccagcgtgctgcgtgcggacagcgagcgaacacacgacggagcagctacgacgaacgggggaccgagtggaccggacgaggatgtggcctaggacgagtgcacaaggctagtggactcggtccccgcgcggtatcccgagtggtccactgtctgcaaacacgattcacatagagcgggcagacgcgggagccgtcctaggtgcaccggaagcaaatccgtcgcctgggtggatttgagtgacacggcccacgtgtagcctcacagctctccgtggtcagatgtgtaaaattatcataatatgtgtttttcaaatagttaaataatatatataggcaagttatatgggtcaataagcagtaaaaaggcttatgacatggtaaaattacttacaccaatatgccttactgtctgatatattttacatgacaacaaagttacaagtacgtcatttaaaaatacaagttacttatcaattgtagtgtatcaagtaaatgacaacaaacctacaaatttgctattttgaaggaacacttaaaaaaatcaataggcaagttatatagtcaataaactgcaagaaggcttatgacatggaaaaattacatacaccaatatgctttattgtccggtatattttacaagacaacaaagttataagtatgtcatttaaaaatacaagttacttatcaattgtcaagtaaatgaaaacaaacctacaaatttgttattttgaaggaacacctaaattatcaaatatagcttgctacgcaaaatgacaacatgcttacaagttattatcatcttaaagttagactcatcttctcaagcataagagctttatggtgcaaaaacaaatataatgacaaggcaaagatacatacatattaagagtatggacagacatttctttaacaaactccatttgtattactccaaaagcaccagaagtttgtcatggctgagtcatgaaatgtatagttcaatcttgcaaagttgcctttccttttgtactgtgttttaacactacaagccatatattgtctgtacgtgcaacaaactatatcaccatgtatcccaagatgcttttttattgctatataaactagcttggtctgtctttgaactcacatcaattagcttaagtttccataagcaagtacaaatagct

SEQ ID NO: 18 - DNA, 638 bp, Oryza sativa
taagagctctgtaattgagaactagtatcggcgtagagtaaaataaaacaccacaagtatgacacttggtggtgattctgttcgatatcagtactaaataaaggttacaaacttcttaattttcctacttcatgccatggatattccattatggactatagtggacagggccggtctatgattttgagggccctaggaactcatcgcgatgggcctcaagctatatataaaatttattgatatatatagacgctaattttacttgcaaaatgaaaacaaatacatctatatattaaatttaacattcctggtaattatcaagaaataaaatcgaccaaaataacaatatatttgtaacttggaactaatataattatttattaacttaatgaagaatagaaccccgtcatatccattgcttcctatgaaaagatacttcttcgggtatttcttgatgcaaaatcataaagaacggtattaagatcaatagtgtccaagatatccttctcgattgagcacatagccaagccatttaaccttatttgcgacagttgatctcaaatagtttttcaacaacttcaattttgataaacttatttcagctgaagctaccatcataggtaaagttaagagaattc

SEQ ID NO: 19 - DNA, 57 bp, Human Immunodeficiency Virus
  CDS (1)..(57)
aatactcgtaagtctattcatattggccctggccgtgctttctatgctactggctct

SEQ ID NO: 20 - DNA, 18 bp, Artificial Sequence
  Description of Artificial Sequence DNA coding hinge peptide sequence and restriction enzyme site
  CDS (1)..(18)
ggaccaggaccaggatcc

SEQ ID NO: 21 - DNA, 1358 bp, Clostridium botulinum
  CDS (3)..(1349)
ccatgggctctgctcgtctcctctctactttcactgagtacatcaagaacatcatcaacacttctattctcaatctccgttatgaatctaatcatctcattgatctctctcgttatgcttctaagattaatattggctctaaggttaatttcgatcctattgataagaatcaaattcaactcttcaatctcgaatcttctaagattgaagttattctcaagaatgctattgtttataatagtatgtatgaaaatttcagtacttctttctggattcgtattccaaagtatttcaatagtatttctctcaataacgaatatactattattaattgtatggaaaataattctggctggaaggtttctctcaattatggcgaaattatttggactctccaagatactcaagaaattaagcagcgtgttgttttcaagtactctcagatgatcaatatcagtgattacatcaatcgttggatcttcgttactatcactaataatcgtctcaataatagtaagatttacatcaatggccgtctcatcgatcaaaagcccatcagtaatctcggcaatatccatgcaagtaataatatcatgttcaagctcgatggctgccgtgatactcatcgttacatctggatcaagtacttcaatctcttcgataaggaactcaatgaaaaggaaatcaaggatctctacgataatcaaagtaacagtggcatcctcaaggatttctggggcgattatctccaatatgataagccatactacatgctcaatctctacgatccaaataagtacgttgatgttaataatgttggcatccgtggctacatgtacctcaagggtccccgtggtagtgttatgactactaatatttatctcaatagtagtctctaccgtggtactaagttcatcatcaagaagtatgcaagtggcaacaaggataatatcgttcgtaataacgatcgtgtttacatcaatgttgttgttaagaataaggaataccgtctcgcaactaatgctagtcaagctggcgttgaaaagattctcagtgctctcgaaatcccagatgttggcaacctcagtcaagttgttgttatgaagagtaagaacgatcaaggcatcactaataagtgcaagatgaatctccaggataataatggcaacgatattggcttcatcggcttccatcaattcaataatatcgcaaagctcgttgctagtaattggtacaatcgtcagatcgagcgtagtagtcgtactctcggctgtagttgggagttcatcccagttgatgatggctggggcgaacgtccactcacaagtagtccaggcaaggacgagttgtaagagctc

SEQ ID NO: 22 - protein, 449 aa, Clostridium botulinum
MGSARLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPLTSSPGKDEL

(19) United States
(12) Patent Application Publication
(10) Pub. No.: US 2007/0192906 A1
(43) Pub. Date: Aug. 16, 2007

(54) Rice plant having vaccine gene transferred thereinto

(76) Inventors: Yoshikazu Yuki, Tokyo (JP); Hiroshi Kiyono, Tokyo (JP); Takachika Hiroi, Kanagawa (JP); Tomonori Nochi, Tokyo (JP); Fumio Takaiwa, Ibaraki (JP); Hidenori Takagi, Ibaraki (JP); Lijyun Yang, Ibaraki (JP); Kazuya Suzuki, Ibaraki (JP); Hiroyasu Ebinuma, Tokyo (JP); Koichi Sugita, Tokyo (JP); Saori Kasahara, Tokyo (JP)

Correspondence Address: ARMSTRONG, KRATZ, QUINTOS, HANSON & BROOKS, LLP, 1725 K STREET, NW, SUITE 1000, WASHINGTON, DC, 20006, US

(21) Appl. No.: 11/547,910

(22) PCT Filed: Apr. 8, 2005
(86) PCT No.: PCT/JP05/06973

§ 371 Date: Oct. 10, 2006

(30) Foreign Application Priority Data
Apr. 9, 2004 (JP) 2004-116270

(51) Int. Cl. A01H 5/00; A61K 39/00; C12N 15/82

(52) U.S. Cl. 800/288; 800/320.2; 424/184.1

(57) ABSTRACT

To provide a transgenic rice plant that can produce rice which can be used as an “edible vaccine”, i.e., rice capable of inducing a desired immune response when mucosally administered such as an oral administration. There is provided a transgenic rice plant including a genomic DNA, wherein a DNA construct is incorporated into the genomic DNA so that the DNA construct is capable of being expressed, wherein the DNA construct includes a DNA encoding an antigenic protein, and a rice endosperm specific promoter linked upstream thereof.

### TECHNICAL FIELD

The present invention relates to transgenic rice plants, vaccine compositions utilizing rice obtained from the transgenic rice plants or their processed products, and methods for inducing immune responses utilizing the vaccine compositions.

### BACKGROUND ART

Many of severe emerging or reemerging infectious diseases such as influenza, SARS (severe acute respiratory syndrome), AIDS, and tuberculosis are caused through tissues covered with mucosa, such as nasal cavity, oral cavity, upper bronchus, intestinal tract, and genitourinary organ. From such facts, it is clear that defense at mucosal surfaces is most effective for preventing infectious diseases. Defense mechanisms at mucosal surfaces are formed by mucosal immunity in which secretory immune globulin A (S-IgA) plays a major role.

Current immunization method by injection can induce a systemic antigen-specific immune response, however; a mucosal antigen-specific immune response cannot be induced sufficiently. (Czerkinsky C et al. Immunol Rev 170. 197-222. 1999). In contrast, mucosal vaccine can be said as an ideal vaccine in that it can establish antigen specific immunity in both of the mucosal and systemic immune systems.

In 1990, Curtiss et al. succeeded in the induction of mucosal antigen-specific immune response by feeding surface protein SpaA of S. mutans expressed in a tobacco plant (Patent Literature 1). This experiment demonstrated the possibility that the plant expressing an antigenic protein can be used as an “edible vaccine” without any treatment (i.e., without purifying antigenic proteins from plants). “Edible vaccine”, one of mucosal vaccines, can be said as an ideal vaccine in that it can establish antigen specific immunity in both of the mucosal and systemic immune systems (Non-Patent Literature 1).

After the experiment by Curtiss et al., expression of Escherichia coli heat-labile toxin antigen in tobacco, potato or corn (Non-Patent Literature 2, 3, 4), expression of cholera toxin antigen in potato (Non-Patent Literature 5), expression of hepatitis B virus antigen in lettuce or potato (Non-Patent Literature 6, 7), expression of Norwalk virus antigen in tobacco or potato (Non-Patent Literature 8), expression of rabies virus antigen or RS virus antigen in tomato (Non-Patent Literature 9, 10), expression of cytomegalovirus antigen in tobacco (Non-Patent Literature 11), expression of foot-and-mouth disease virus antigen in alfalfa (Non-Patent Literature 12), expression of porcine transmissible gastroenteritis virus antigen in Arabidopsis (Non-Patent Literature 13), etc. were performed.

Even if an antigenic protein can be expressed in plants, the use of the plant expressing the antigenic protein as an “edible vaccine” is different issue. Namely, whether or not the plant expressing the antigenic protein can induce an intended immune response when the plant is orally administered, is determined depending on various factors such as expression level of antigenic protein in plant, tertiary structure of the antigenic protein expressed in plant, and interaction with other components contained in plant. Thus, oral administration of plant expressing the antigenic protein does not always lead directly to the induction of intended immune response. For example, there are cases where intended immune response cannot be induced due to inadequate expression level of antigenic protein in plant, where intended immune response cannot be induced because the tertiary structure of antigenic protein expressed in plant is different from the original tertiary structure (e.g. a case where plant-specific sugar residues such as α1,3-fucose and β1,2-xylose are added, resulting in different tertiary structure), where intended immune response cannot be induced because other components contained in plant, which are inevitably administered together with the antigenic protein, constitute barriers to the induction, etc. Therefore, even in the case where antigen-specific immune response can be induced by the oral administration of an adequate amount of purified antigenic protein, it is extremely difficult to predict whether or not antigen-specific immune response can be induced when the plant expressing the above-mentioned antigenic protein is orally administered.

Factors, which determine whether intended immune response can be induced or not when the plant expressing an antigenic protein is orally administered, such as expression level of antigenic protein in plant, tertiary structure of antigenic protein expressed in plant, and interaction with other components contained in plant, are influenced by various conditions such as the types of plant, expression site of antigenic protein in plant, accumulation site of antigenic protein in plant, and expression system (e.g. promoter) of antigenic protein. Thus, it is extremely difficult to set conditions that allow the induction of intended immune response when the plant expressing an antigenic protein is orally administered.

Patent Literature 1

International Publication No. WO 90/02484

Non-Patent Literature 1

Yuki Y et al., Reviews in Medical Virology, 13:293-310, 2003

Non-Patent Literature 2

Haq T A et al., Science, 268:714-716, 1995

Non-Patent Literature 3

Mason H S et al., Vaccine, 16:1336-1343, 1998

Non-Patent Literature 4

Streatfield S J et al., Vaccine, 19:2742-2748, 2001

Non-Patent Literature 5

Arakawa T et al., Nature Biotechnology, 16:292-297, 1998

Non-Patent Literature 6

Kapusta J et al., The FASEB JOURNAL, 13:1796-1799, 1999

Non-Patent Literature 7

Richter L J et al., Nature Biotechnology, 18:1167-1171, 2000

Non-Patent Literature 8

Mason H S et al., Proceedings of National Academy of Science U S A., 93:5335-5340, 1996

Non-Patent Literature 9

McGarvey P B et al., Biotechnology, 13:1484-1487, 1995

Non-Patent Literature 10

Sandhu J S et al., Transgenic Research, 9:127-135, 2000

Non-Patent Literature 11

Tackaberry E S et al., Vaccine, 17:3020-3029, 1999

Non-Patent Literature 12

Dus Santos M J et al., Vaccine, 20: 1141-1147, 2002

Non-Patent Literature 13

Gomez N et al., Virology, 249:352-358, 1998

### DISCLOSURE OF INVENTION

Problems to Be Solved by the Invention

Conventional vaccines inevitably need to be stored and transported under low-temperature condition, i.e., so-called cold chain is essential.

In addition to this requirement, injection-type vaccine is required to be purified to the level allowing injection from safety reason. Considering storage and transport under low-temperature condition as described above, it is inevitable for conventional vaccines to be expensive in terms of cost.

In contrast, non-recombinant vaccines using an attenuated strain of pathogenic microorganism, which are relatively low-cost and, have problems in terms of safety. For the method in which recombinant bacteria, etc. are allowed to express a vaccine antigen and orally administered, which can be developed at relatively low cost, there is a problem in that vaccine cannot be used to the same individual repeatedly. This is because this method not only induces the expected immune response to vaccine, but also induces a strong immune response to the recombinant microorganism, etc. as a vector, by which the recombinant microorganism itself may be eliminated when administered for additional immunization.

On the other hand, rice can be stored for a long time at room temperature as well as it can be mass produced. In addition, rice can be transported easily to a distant place including developing countries. Thus, development of rice that can be used as an “edible vaccine” will make it possible to store a variety of vaccines at low cost and in large quantities for a long time. Then, such rice will make it possible to achieve vaccine administration on a global scale (global vaccination) and public health on a global scale (global public health).

Accordingly, a first object of the invention is to provide a transgenic rice plant that can produce rice which can be used as an “edible vaccine”, i.e., rice capable of inducing a desired immune response when mucosally administered such as an oral administration.

Further, a second object of the invention is to provide a processed rice product that includes rice or an antigenic protein obtained from the above-mentioned transgenic rice plant.

Further, a third object of the invention is to provide a vaccine composition that includes as an active ingredient the above-mentioned processed rice product including rice or an antigenic protein.

Further, a fourth object of the invention is to provide a method for inducing an immune response utilizing the above-mentioned vaccine composition.

Means for Solving the Problems

The inventions have found the following:
(a) various antigenic proteins (vaccine antigens) having as a monomer a molecular mass of 100,000 dalton or less can be expressed in rice;
(b) antigenic proteins can be expressed in rice at a high level by changing the codon of DNA encoding the antigenic protein (vaccine antigen) to a plant codon (rice codon);
(c) antigenic proteins (vaccine antigens) expressed in rice are stable at room temperature for one year or more;
(d) systemic and mucosal antigen-specific immune response can be induced by mucosal administration, such as oral administration, of the rice expressing an antigenic protein (vaccine antigen);
(e) resistance to pepsin can be provided by allowing antigenic proteins (vaccine antigens) to be expressed in rice;
(f) the oral dosage of antigenic protein needed for inducing the systemic and mucosal antigen-specific immune response is smaller than that of purified antigenic protein since antigenic proteins (vaccine antigens) expressed in rice can escape digestion in stomach;
(g) when antigenic proteins (vaccine antigens) are expressed in rice, the antigenic proteins are accumulated in type I storage protein body (protein body I), type II storage protein body (protein body II), etc. in a rice endosperm cell. In order to provide the antigenic proteins with resistance to pepsin, accumulation of the antigenic proteins in type I storage protein body (protein body I) in rice endosperm cell is important; and
(h) the antigenic proteins (vaccine antigens) expressed in rice reach the intestinal tract without being digested in the stomach and are taken up by cells (for example, M cells) that are present in e.g. a Peyer's patch which is an mucosal inductive tissue and that are capable of taking up antigen, thereby allowing the induction of antigen-specific immune response etc., which led to the completion of the invention. Specifically, in order to achieve the above-mention objects, the invention provides the following transgenic rice, rice or processed rice product, vaccine composition, and method for inducing an immune response.
(1) A transgenic rice plant including a genomic DNA, wherein a DNA construct is incorporated into the genomic DNA so that the DNA construct is capable of being expressed, wherein the DNA construct includes a DNA encoding an antigenic protein, and a rice endosperm specific promoter linked upstream thereof.
(2) The transgenic rice plant according to the (1), wherein the antigenic protein as a monomer has a molecular mass of 100,000 dalton or less.
(3) The transgenic rice plant according to one of the (1) and (2), wherein the antigenic protein is a subunit constituting a homomultimer.
(4) The transgenic rice plant according to the (3), wherein the homomultimer has a molecular mass of 1,000,000 dalton or less.
(5) The transgenic rice plant according to one of the (1) and (2), wherein the antigenic protein is a subunit constituting a heteromultimer.
(6) The transgenic rice plant according to the (5), wherein the DNA construct includes a DNA encoding each subunit constituting the heteromultimer.
(7) The transgenic rice plant according to the (6), wherein the heteromultimer has a molecular mass of 1,000,000 dalton or less.
(8) The transgenic rice plant according to one of the (1) and (2), wherein the antigenic protein is one of a cholera toxin B subunit, a fusion protein of a cholera toxin B subunit and a neutralizing epitope of AIDS virus, and an Hc domain of botulinum toxin.
(9) The transgenic rice plant according to any one of the (1) to (8), wherein the rice endosperm specific promoter is a promoter of a gene encoding a storage protein in rice endosperm.
(10) The transgenic rice plant according to the (9), wherein the gene encoding a storage protein in rice endosperm is one of a glutelin gene, a prolamin gene and a globulin gene.
(11) The transgenic rice plant according to the (10), wherein the glutelin gene is glutelin GluB-1 gene.
(12) The transgenic rice plant according to any one of the (1) to (11), wherein the DNA construct includes a rice endosperm specific terminator which is linked downstream of the DNA encoding an antigenic protein.
(13) The transgenic rice plant according to the (12), wherein the rice endosperm specific terminator is a terminator of a gene encoding a storage protein in rice endosperm.
(14) The transgenic rice plant according to the (13), wherein the gene encoding a storage protein in rice endosperm is one of a glutelin gene, a prolamin gene and a globulin gene.
(15) The transgenic rice plant according to any one of the (1) to (14), wherein the DNA construct includes a DNA encoding a signal peptide of a storage protein in rice endosperm so that a fusion protein is produced by the expression of the DNA construct, wherein the DNA encoding a signal peptide is linked upstream of the DNA encoding an antigenic protein, wherein the fusion protein includes the antigenic protein and the signal peptide attached to the N terminus thereof.
(16) The transgenic rice plant according to the (15), wherein the signal peptide of a storage protein in rice endosperm is a signal peptide of one of glutelin, prolamin and globulin.
(17) The transgenic rice plant according to any one of the (1) to (16), wherein the DNA construct includes a DNA encoding an endoplasmic reticulum-localization signal peptide so that a fusion protein is produced by the expression of the DNA construct, wherein the DNA encoding an endoplasmic reticulum-localization signal peptide is linked downstream of the DNA encoding an antigenic protein, wherein the fusion protein includes the antigenic protein and the endoplasmic reticulum-localization signal peptide attached to the C terminus thereof.
(18) The transgenic rice plant according to the (17), wherein the endoplasmic reticulum-localization signal peptide includes an amino acid sequence described in sequence number 2.
(19) The transgenic rice plant according to any one of the (1) to (18), wherein at least one codon contained in the DNA encoding an antigenic protein is modified to a plant codon.
(20) A processed rice product, including one of rice and the antigenic protein which are obtained from the transgenic rice plant of any one of the (1) to (19).
(21) A vaccine composition, including as an active ingredient the processed rice product of the (20) which includes one of rice and the antigenic protein.
(22) A method for inducing an immune response in an animal other than a human, including mucosally administering the vaccine composition of the (21) to the animal other than a human.
(23) A method for inducing an immune response in a human, comprising mucosally administering the vaccine composition of the (21) to the human.

### EFFECT OF THE INVENTION

In a first aspect of the invention, a transgenic rice plant is provided that can produce rice which can be used as an “edible vaccine”, i.e., rice capable of inducing a desired immune response when mucosally administered such as an oral administration. Further, in a second aspect of the invention, a processed rice product is provided that includes rice or an antigenic protein obtained from the above-mentioned transgenic rice plant. Further, in a third aspect of the invention, a vaccine composition is provided that includes as an active ingredient the above-mentioned processed rice product including rice or an antigenic protein. Further, in a fourth aspect of the invention, a method for inducing an immune response utilizing the above-mentioned vaccine composition is provided.

### BEST MODE FOR CARRYING OUT THE INVENTION

The “rice plant” means plants belong to Oryza sativa, and the variety of rice plant served as a subject to be genetically modified is not particularly limited as long as they belong to Oryza sativa.

The “antigenicity” includes characteristics to induce antibody production and cellular immunity (immunogenicity) and characteristics to allow an antigen-antibody reaction and cellular immune response to occur, and “antigenic protein” means proteins having at least immunogenicity. The immunogenicity, which the antigenic protein has, is enough if the immunogenicity is achieved when the antigenic protein is administered singly or together with an immune adjuvant to humans or other animals through any one of administration routes such as intravenous, interperitoneal, subcutaneous, intracutaneous, oral, nasal, vaginal, anal, and pulmonary. The “immune adjuvant” refers to an antigenicity strengthening agent which enhances antigenicity of proteins.

The type of the antigenic protein are not particularly limited as long as the antigenic protein can induce immune response in vivo when the antigenic protein is administered singly or together with an immune adjuvant to a human or other animals through any one of administration routes such as intravenous, interperitoneal, subcutaneous, intracutaneous, oral, nasal, vaginal, anal, and pulmonary. It is enough if the antigenic protein can induce an immune response when an administration form, dosage, administration route, and the like are set to most favorable conditions (for example, when an adequate amount of purified antigenic protein is administered). The “immune response” includes a variety of immune responses such as antibody production, antigen-antibody reaction, and cellular immune response, but “immune response” used in the invention includes at least antibody production. The “animal” includes humans and other vertebrates (e.g. mammals, birds, amphibians, fishes, reptiles). The “mucosal administration” includes oral administration, intraoral administration, intranasal administration, anal administration, vaginal administration, pulmonary administration, and the like. The “antigenic protein” also includes glycoproteins.

Examples of the antigenic protein include proteins which are derived from pathogenic microorganisms, which themselves are not pathogenic or toxic, or weakly pathogenic or toxic to such an extent not to adversely affect the living body, but which retain immunogenicity.

The type of the pathogenic microorganism is not particularly limited. Examples thereof include pathogenic bacteria such as cholera vibrio, pathogenic Escherichia coli, Haemophilus influenzae, a pneumococcus, Bordetella pertussis, diphtheria bacillus, plague bacillus, tetanus bacillus, Clostridium botulinum, anthrax bacillus, Francisella tularensis, Escherichia coli 0157, salmonella, MRSA (Staphylococcus aureus), VRE (enterococcus), tubercle bacillus, dysentery bacillus, typhoid bacillus, Salmonella paratyphi, chlamydia, amebic dysentery, legionella, borrelia causing Lyme disease, and brucella causing brucellosis (undulant fever); pathogenic viruses such as a rotavirus, hepatitis A virus, hepatitis B virus, hepatitis C virus, Norwalk virus, rabies virus, RS virus, cytomegalovirus, foot-and-mouth disease virus, transmissible gastroenteritis virus, rubella virus, ATL virus, adenovirus, mumps (epidemic parotiditis) virus, Coxsackie virus, enterovirus, herpesvirus, smallpox virus, poliovirus, measles virus, Japanese encephalitis virus, dengue fever virus, yellow fever virus, West Nile virus, SARS (coronavirus), influenza virus, HIV (AIDS virus), Ebora fever virus (filovirus), Marburg virus (filovirus), Lassa fever virus, hantavirus, and Nipah virus; rickettsiae such as a Q fever rickettsia and chlamydia; and protozoa such as a plasmodium and trypanosome. Examples of the protein which is derived from a pathogenic microorganism include proteins or peptides which are constituents of the pathogenic microorganism (e.g. a surface protein, capsid protein, pilus protein), proteins or peptides produced by the pathogenic microorganism (e.g. a toxin, enzyme, hormone, immunomodulating substance, receptor and its ligand), fragments or domains thereof, and the like.

Examples of the proteins which are derived from pathogenic microorganisms, which themselves are not pathogenic or toxic, or weakly pathogenic or toxic to such an extent not to adversely affect the living body, but which retain immunogenicity include cholera toxin B subunit (CTB), Escherichia coli heat-labile toxin B subunit (LTB), anthrax bacillus protective antigen, tetanus toxin ToxC domain, botulinum toxin Hc domain, influenza virus HA antigen, Yersinia pestis F1 antigen or V antigen, malaria SERA antigen, rotavirus VP6 or VP7; gp-160, nef, gag, env or tat proteins derived from AIDS virus, S proteins of hepatitis B virus, and the like.

The antigenic protein may be a fusion protein (chimeric protein) composed of two or more different proteins or peptides. Examples of the fusion protein include fusion proteins consisting of CTB or LTB having an action such as regulation and enhancement of mucosal immune response and other antigenic protein or its epitope (e.g. T-cell epitope, B-cell epitope, neutralizing epitope).

The molecular mass of the antigenic protein as a monomer is not particularly limited, typically 100,000 dalton or less, preferably 80,000 dalton or less, more preferably 50,000 dalton or less. The antigenic protein with molecular mass as a monomer within the above-mentioned range can be expressed in a rice endosperm cell without losing its antigenicity, and when rice obtained from transgenic rice plant, or its processed product is mucosally administered to humans or other animals, systemic and mucosal immune responses to the antigenic protein can be effectively induced in vivo. The lower limit of the molecular mass of the antigenic protein as a monomer is not particularly limited, typically 1,000 dalton or more, preferably 3,000 dalton or more, more preferably 5,000 dalton or more.

When the antigenic protein is a subunit constituting a homomultimer, expression of the antigenic protein in a rice endosperm cell results in the formation of homomultimer. In this case, the molecular mass of the homomultimer is typically 1,000,000 dalton or less, preferably 600,000 dalton or less, more preferably 300,000 dalton or less. If the molecular mass of homomultimer is within the above-mentioned range, systemic and mucosal immune responses to the homomultimer can be effectively induced in vivo when rice obtained from transgenic rice plant, or its processed product is mucosally administered to humans or other animals.

When the antigenic protein is a subunit constituting a heteromultimer, expression of each subunit constituting the heteromultimer in a rice endosperm cell results in the formation of heteromultimer. In this case, the molecular mass of the heteromultimer is typically 1,000,000 dalton or less, preferably 600,000 dalton or less, more preferably 300,000 dalton or less. If the molecular mass of heteromultimer is within the above-mentioned range, systemic and mucosal immune responses to the heteromultimer can be effectively induced in vivo when rice obtained from transgenic rice plant, or its processed product is mucosally administered to humans or other animals.

Examples of antigenic protein comprising a homomultimer include proteins such as cholera toxin B subunit pentamer (CTB5), Escherichia coli heat-labile toxin B subunit pentamer (LTB5), and anthrax bacillus protective antigen heptamer (PA7). Examples of antigenic protein comprising a heteromultimer include a nontoxic mutated form of cholera toxin (mCTA-CTB5) and a nontoxic chimeric mutated form of toxin (mCTA-LTB5).

The nontoxic mutated form of cholera toxin is comprised of: S61F in which Ser (S) at position 61, an ADP ribosyltransferase active center of cholera toxin A subunit with toxic activity, is substituted with Phe (F), or E112K in which Glu (E) at position 112 is substituted with Lys (K); and cholera toxin B subunit, and the nontoxic mutated form of cholera toxin is nontoxic, but retains mucosal immune adjuvant activity which cholera toxin possess (Yamamoto S et al. J Exp Med. 185:1203-10 (1997) ; Yamamoto S et al. Proc Natl Acad Sci USA. 94:5267-72 (1997)).

The nontoxic chimeric mutated form of toxin is comprised of the above-mentioned E112K, a nontoxic A subunit of cholera toxin, and instead of cholera toxin B subunit, an Escherichia coli heat-labile toxin (LT) B subunit (LTB) having immune adjuvant activity similar to that of the cholera toxin B subunit. The chimeric mutated form of toxin is nontoxic, but has mucosal immune adjuvant activity (Kweon MN et al. J Infect Dis. 2002 186:1261-9 (2002)).

The antigenic protein may be a native protein or may be a mutated form of a protein as long as the antigenic protein possesses desired antigenicity. The “mutated form of a protein” means a protein that has an amino acid sequence in which one or more amino acids of the amino acid sequence of the native protein are substituted or deleted, or one or more amino acids are added to the amino acid sequence of the native protein, and that possesses antigenicity similar to that of the native protein.

The antigenic protein may form a fusion protein together with other protein or peptide as long as the antigenic protein possesses desired antigenicity.

Examples of the fusion protein include fusion proteins that contain an antigenic protein and a signal peptide of storage protein in rice endosperm, which is attached to the N terminus of the antigenic protein. When the antigenic protein has the signal peptide of storage protein in rice endosperm, the antigenic protein expressed in the rice endosperm cell can be efficiently accumulated in the rice endosperm cell, allowing the accumulation of antigenic protein in the rice endosperm cell sufficient to induce an immune response.

Examples of the signal peptide of storage protein in rice endosperm include signal peptides of glutelin, prolamin, or globulin. As a specific example of the signal peptide of storage protein in rice endosperm, the amino acid sequence of the signal peptide of glutelin GluB-1, and the base sequence of DNA that encodes the amino acid sequence are shown in sequence numbers 16 and 15, respectively. Signal peptides of glutelin, prolamin, or globulin are involved in the accumulation in storage protein body in the rice endosperm cell. When the antigenic protein has a signal peptide of glutelin, prolamin, or globulin, the antigenic protein can be efficiently accumulated in storage protein body (protein bodies I, II) or the like in the rice endosperm cell. In the storage protein body (protein bodies I, II) in the rice endosperm cell, sufficient amount of antigenic protein to induce an immune response is accumulated without losing its antigenicity, and when the rice obtained from the transgenic rice plant, or its processed product is mucosally administered to humans or other animals, systemic and mucosal immune responses to the antigenic protein can be effectively induced in vivo.

Examples of fusion protein further include fusion proteins that contain an antigenic protein and an endoplasmic reticulum-retention signal peptide, which is attached to the C terminus of the antigenic protein. When the antigenic protein has the endoplasmic reticulum-retention signal peptide, the antigenic protein expressed in the rice endosperm cell can be efficiently accumulated in storage protein body (protein bodies I, II) or the like in the rice endosperm cell, allowing the accumulation of antigenic protein in the rice endosperm cell sufficient to induce an immune response. Examples of the endoplasmic reticulum-retention signal peptide include a peptide that has an amino acid sequence KDEL (sequence number 2), and a peptide that has an amino acid sequence HDEL (His-Asp-Glu-Leu).

Examples of fusion protein further include fusion proteins that contain an antigenic protein, a signal peptide of storage protein in rice endosperm, which is attached to the N terminus of the antigenic protein, and an endoplasmic reticulum-retention signal peptide, which is attached to the C terminus of the antigenic protein. When the antigenic protein has the signal peptide of storage protein in rice endosperm and endoplasmic reticulum-localization signal peptide, the antigenic protein expressed in the rice endosperm cell can be accumulated more efficiently in storage protein body (protein bodies I, II) or the like in the rice endosperm cell, allowing the accumulation of antigenic protein in the rice endosperm cell sufficient to induce an immune response.

It is preferable that the codon contained in the DNA that encodes the antigenic protein be modified to a plant codon. Modification to the plant codon can improve expression efficiency of the antigenic protein in the rice endosperm cell, allowing the expression of antigenic protein in the rice endosperm cell sufficient to induce an immune response.

The “rice endosperm specific promoter” and “rice endosperm specific terminator” mean a promoter and terminator which function specifically in a rice endosperm cell, and the types thereof are not particularly limited. Examples of the rice endosperm specific promoter and terminator include promoters and terminators of genes that encode storage proteins in rice endosperm. Utilization of the promoter and terminator of gene that encodes a storage protein in rice endosperm enables efficient expression of the antigenic protein in the rice endosperm cell, allowing the accumulation of antigenic protein sufficient to induce an immune response, in storage protein body (protein bodies I, II), etc. in the rice endosperm cell.

The “storage protein in rice endosperm” means a protein which is specifically expressed in a rice endosperm cell and accumulated in the rice endosperm cell, and the types thereof are not particularly limited. Examples of the storage protein in rice endosperm include glutelin, prolamin, and globulin.

The promoter of the gene encoding the storage protein in rice endosperm is preferably a promoter of glutelin GluB-1 gene. Higher promoter activity of the promoter of glutelin GluB-1 gene than those of other promoters of genes encoding the storage protein in rice endosperm enables efficient expression of the antigenic protein in the rice endosperm cell, allowing the accumulation of antigenic protein sufficient to induce an immune response, into storage protein body (protein bodies I, II), etc. in the rice endosperm cell.

The rice endosperm specific promoter may be a native promoter or may be a mutated form of a promoter as long as it has rice endosperm specific promoter activity. The “mutated form of a promoter” means a promoter that has a base sequence in which one or more bases of the base sequence of the native promoter are substituted or deleted, or one or more bases are added to the base sequence of the native promoter, and that has rice endosperm specific promoter activity. This is also true for the rice endosperm specific terminator.

As a specific example of the native promoter and terminator, base sequences of the promoter and terminator of rice glutelin GluB-1 gene are shown in sequence numbers 17 and 18, respectively. Specific examples of the mutated form of a promoter include promoters that have a base sequence in which one or more bases of the base sequence of sequence number 17 are substituted or deleted, or one or more bases are added to the base sequence of sequence number 17, and that have rice endosperm specific promoter activity. Specific examples of the mutated form of a terminator include terminators that have a base sequence in which one or more bases of the base sequence of sequence number 18 are substituted or deleted, or one or more bases are added to the base sequence of sequence number 18, and that have rice endosperm specific terminator activity.

A DNA construct, to be incorporated into the rice genomic DNA so that it can be expressed, includes DNA that encodes the antigenic protein and the rice endosperm specific promoter that is linked to the upstream thereof.

When the antigenic protein is a subunit constituting a heteromultimer, the DNA construct preferably comprises a DNA that encodes each subunit constituting the heteromultimer. This allows the formation of heteromultimer in the rice endosperm cell.

When the fusion protein is expressed that contains the antigenic protein and the signal peptide of storage protein in rice endosperm, which is attached to the N terminus of the antigenic protein, the DNA construct comprises a DNA that encodes the signal peptide and is linked upstream of a DNA encoding the antigenic protein. The DNA that encodes the signal peptide is linked downstream of the rice endosperm specific promoter.

When the fusion protein is expressed that contains the antigenic protein and the endoplasmic reticulum-localization signal peptide, which is attached to the C terminus of the antigenic protein, the DNA construct comprises a DNA that encodes the endoplasmic reticulum-localization signal peptide and is linked downstream of a DNA encoding the antigenic protein. The DNA that encodes the endoplasmic reticulum-localization signal peptide is linked upstream of the rice endosperm specific terminator.

The method for generating a transgenic rice plant, in which the DNA construct is incorporated into the genomic DNA so that it can be expressed, is not particularly limited. The transgenic rice plant can be generated, for example, by introducing a vector containing the DNA construct into a rice cell, and then cultivating a transformed rice cell to thereby allow rice plants to regenerate. The rice cell into which a vector is introduced may be any form without limitation as long as the rice cell can be regenerated to plants. Examples of the form of the rice cell include cultured cells, protoplasts, shoot primordia, multiple shoots, hairy roots, and calli. When a plasmid is used as a vector, it is preferable that the plasmid comprise a drug resistance gene such as hygromycin, tetracycline, and ampicillin so that the rice cell, into which the vector is introduced, can be efficiently selected. The vector can be introduced into the rice cell by any method without limitation, and examples thereof include indirect introduction methods using Agrobacterium tumefaciens, Agrobacterium rhizogenes, or the like (Hiei, Y. et al., Plant J., 6, 271-282, 1994; Takaiwa, F. et al., Plant Sci. 111, 39-49, 1995; Japanese Patent (JP-B) No. 3141084); or direct introduction methods such as an electroporation method (Tada, Y. et al. Theor. Appl. Genet, 80, 475, 1990), polyethylene glycol method (Datta, S. K. et al., Plant Mol Biol., 20, 619-629, 1992), and particle gun method (Christou, P. et al., Plant J. 2, 275-281, 1992; Fromm, M. E., Bio/Technology, 8, 833-839, 1990). The method for regenerating rice plants by cultivating rice cells is not particularly limited and may be performed according to the method known in the art.

Since the antigenic protein sufficient to induce an immune response is accumulated in the endosperm cell of the transgenic rice plant without losing its antigenicity, mucosal administration of the rice obtained from the transgenic rice plant or its processed product to humans or other animals enables induction of systemic and mucosal immune responses in vivo. Therefore, the rice obtained from the transgenic rice plant or its processed product can be used as an active ingredient of vaccine composition, and by mucosally administering the vaccine composition to humans or other animals, a desired immune response can be induced in vivo. In addition, the accumulation of antigenic protein in the rice endosperm cell (especially, protein body I) provides the antigenic protein with resistance to pepsin. Thus, the antigenic protein accumulated in the rice endosperm cell (especially, protein body I) reaches the intestinal tract without being digested in the stomach and is taken up by cells (for example, M cells) that are present in e.g. a Peyer's patch, an mucosal inductive tissue and that are capable of taking up antigen, thereby allowing the induction of antigen-specific immune response. Thus, the amount of antigenic protein to be administered orally, required for inducing systemic and mucosal antigen-specific immune responses, is smaller than that of purified antigenic protein. “Rice” means a tissue containing endosperm, or a portion thereof and includes unhulled rice, unpolished rice, seeds, polished rice, and portions of these. “Processed rice product” includes any processed products as long as they contain the antigenic protein. Examples of processing to be added to rice include threshing, powderization, extraction of protein fraction, and purification of extracted protein fraction.

Examples of the animal, to which rice or its processed product is administered, include humans and other vertebrates (e.g. mammals, birds, amphibians, fishes, reptiles). Examples of mucosal administration include oral administrations, intraoral administrations, and intranasal administrations.

The form of the vaccine composition can be appropriately selected depending on e.g. an administration route. The form of the vaccine composition includes pharmaceutical compositions, food and drink compositions, and feed compositions. Compositions with a variety of forms such as a pharmaceutical composition, food and drink composition, and feed composition can be prepared according to a common method. The vaccine composition may be administered together with an appropriate adjuvant.

The amount of the vaccine composition to be administered is appropriately set depending on the conditions such as age, sex, body weight and symptom of the animal to which the vaccine composition is administered, and administration route, but in general, the amount is in a range of 1 μg/kg body weight per day to 3,000 μg/kg body weight per day, preferably 5 μg/kg body weight per day to 1,000 μg/kg body weight per day, most preferably 25 μg/kg body weight per day to 400 μg/kg body weight per day, each in terms of the amount of antigenic protein to be administered. The above-mentioned dosage may be administered every several days, or may be administered every several weeks to every several months, for example, every one to twelve weeks, or every one to twelve months. The appropriate number of administration is several times to several dozen times, but the number is not particularly limited.

### EXAMPLE

Hereinafter, the invention will be described referring to Examples. In the Examples below, detailed experimental procedures of genetic engineering technique were performed according to Molecular Cloning, Second Edition (Sambrook et al. eds. Cold Spring Harbor Laboratory Press, New York 1989) or instructions provided by manufacturers unless otherwise stated.

### Example 1

### Construction of T-DNA vector into Which Cholera Toxin Subunit B (CTB) Gene is Incorporated

Two types of CTB gene, native CTB gene (nCTB gene) and plant codon-optimized CTB gene (mutated form of CTB, mCTB gene), were prepared.

The native CTB gene (nCTB gene) was amplified by PCR using cholera toxin (CT) gene of V. cholera (Sanchez, J and Holmgren, J. (1989) Proc. Natl. Acad. Sci. USA, vol 86, pp 481-485) as a template. PCR was performed using a forward primer CTB-F-NcoI (sequence number 3) and reverse primer CTB-KDEL-R (sequence number 4), and as a PCR product, nCTB-KDEL gene, in which DNA (sequence number 1) encoding KDEL (sequence number 2) was added to the 3′ end of nCTB gene, was obtained.

The codon of nCTB gene was modified to plant codon (particularly, codon of rice) and the codon was optimized for expression in rice. Specifically, among the codons of nCTB gene, twelve codons that are hardly utilized in plant were modified to plant codon (Table 1) to thereby prepare a mutated form of CTB gene (mCTB gene: sequence number 5). The amino acid sequence of CTB, which is encoded by the nCTB gene and mCTB gene, is shown in sequence number 6.

TABLE 1

| Codon before | Codon after |
|---|---|
| modification | modification |
| ttt | ttc |
| tta | cta |
| cta | ctc |
| ata | atc |
| gtc | gtt |
| tcg | tct |
| acg | act |
| gcg | gca |
| cac | cat |
| caa | cag |
| aaa | aag |
| ggt | ggc |

The mCTB gene consists of two fragments (F1, F2) with a length of about 170 bps. The two DNA fragments were prepared by PCR using as a template a plasmid in which annealed complementary oligo-DNA pairs (for F1, an oligo-DNA pair consisting of F1-up (sequence number 7) and F1-low (sequence number 8), and for F2, an oligo-DNA pair consisting of F2-up (sequence number 9) and F2-low (sequence number 10)) were cloned. In the PCR, a forward primer F1-Fd (sequence number 11) and a reverse primer F1-Rv (sequence number 12) were used as the primer for amplifying F1, and a forward primer F2-Fd (sequence number 13) and a reverse primer F2-Rv (sequence number 14) were used as the primer for amplifying F2.

PCR was performed using mCTB gene as a template, and the forward primer F1-Fd (sequence number 11) and reverse primer CTB-KDEL-R (sequence number 4). As a PCR product, mCTB-KDEL gene, in which DNA (sequence number 1) encoding KDEL (sequence number 2) was added to the 3′ terminus of mCTB gene, was obtained.

The nCTB-KDEL gene or mCTB-KDEL gene, which was obtained as a PCR product, was subcloned by inserting each of the genes between the DNA (sequence number 15) encoding a signal peptide of rice glutelin GluB-1 (sequence number 16) and the terminator of rice glutelin GluB-1 gene (0.6 kbs) (sequence number 18). The DNA (sequence number 15) was linked downstream of a promoter (2.3 kbs) (sequence number 17) of rice glutelin GluB-1 gene, and in the subcloning, restriction enzymes, NcoI and SacI were used. As mentioned above, a DNA construct was prepared that contains the promoter of rice glutelin GluB-1 gene, the DNA that is linked downstream thereof and encodes a signal peptide of rice glutelin GluB-1, nCTB gene or mCTB gene that is linked downstream thereof, the DNA that is linked downstream thereof and encodes KDEL, and the terminator of rice glutelin GluB-1 gene, which terminator is linked downstream thereof. This DNA construct was introduced, as a HindIII/EcoRI fragment, into pGPTV-35S-HPT that contains a hygromycin B resistance gene.

In this way, a T-DNA vector, pGPTV-35S-HPT-2.3kGluBpro-sig-mCTB, into which the mCTB gene is incorporated (See FIG. 1), and a T-DNA vector, pGPTV-35S-HET-2.3kGluBpro-sig-nCTB, into which the nCTB gene is incorporated (See FIG. 1), were constructed.

### Example 2

### Introduction of T-DNA Vector into Agrobacterium

Agrobacterium tumefaciens strain EHA105 was inoculated into 10 mL of YEA liquid medium (beef extract 5 g/L, yeast extract 1 g/L, sucrose 5 g/L, 2 mM MgSO₄) (pH 7.2), and was cultured at 28° C. until OD 630 nm reached the range of 0.4 to 0.6. The culture medium was centrifuged at 6900×g and 4° C. for 10 minutes and cells were collected. Then, the cell pellet was suspended in 20 mL of 10 mM HEPES (pH 8.0), and centrifuged again to collect cells. Subsequently, the collected cells were suspended in liquid YEB medium to obtain a bacterial suspension for introducing a plasmid.

The T-DNA vector pGPTV-35S-HPT-2.3kGluBpro-sig-mCTB or pGPTV-35S-HPT-2.3kGluBpro-sig-nCTB was introduced into Agrobacterium by an electroporation method. An electrical pulse with 2.5 kV, 25μ, 200 ω was applied to 50 μL of bacterial suspension for introducing a plasmid, mixed with 3 μg of each plasmid with an interelectrode distance of 0.2 cm, and each plasmid was introduced into Agrobacterium. An electrical pulse was applied by means of Genepulser II, manufactured by Bio-Rad Laboratories, Inc.

After the electroporation, cells were added to 0.2 mL of liquid YEB medium and cultured with shaking at 28° C. for one hour. Then, the medium was plated on solid YEB medium added with 50 mg/L of kanamycin and cultured at 28° C. for two days to thereby select strains into which a plasmid was introduced. Whether plasmid was introduced or not was finally confirmed by cutting with restriction enzyme the plasmid extracted from the selected strain by alkaline SDS method and by comparing electrophoretic patterns of the cleavage fragments.

### Example 3

### Generation of CTB-Transgenic Rice Plants

The Agrobacterium tumefaciens strain EHA105, into which pGPTV-35S-HPT-2.3kGluBpro-sig-mCTB or pGPTV-35S-HPT-2.3kGluBpro-sig-nCTB had been introduced, that was selected in Example 2 was cultured again on solid YEB medium at 28° C. for two days, and then cultured overnight in liquid YEB medium at 25° C. and at 180 rpm. The culture was centrifuged at 300 rpm for twenty minutes to collect cells. Then, the cells were suspended in N6 liquid medium containing 10 mg/L acetosyringone, 2 mg/L 2,4-D (2,4-dichlorophenoxyacetic acid), and 30 g/L sucrose so that OD 600 nm was 0.1 to obtain an Agrobacterium suspension for infection.

CTB-transgenic rice plants were generated according to JP-B No. 3141084, by infecting fully ripened seeds of rice with the Agrobacterium tumefaciens strain EHA105 into which pGPTV-35S-HPT-2.3kGluBpro-sig-mCTB or pGPTV-35S-HPT-2.3kGluBpro-sig-nCTB had been introduced. Specifically, the CTB-transgenic rice plants were generated by the following method.

(1) Sterilization

After the chaff of rice seed, from Oryza sativa cv. Kita-ake, was removed, the rice seeds in an intact state were sterilized in 2.5% sodium hypochlorite (NaClO) solution. After the rice seeds were washed with water well, they were subjected to the following aseptic manipulations.

(2) Preculture

Rice seeds were inoculated into 2,4-D containing N6D medium (30 g/l sucrose, 0.3 g/l casamino acid, 2.8 g/l proline, 2 mg/l 2,4-D, 4 g/l Gel-rite, pH 5.8), and incubated at 27° C. to 32° C. for five days. During this period, the rice seeds germinated.

(3) Agrobacterium Infection

Pre-cultured rice seeds were immersed in the Agrobacterium suspension for infection, and then transferred to 2N6-AS medium (30 g/L sucrose, 10 g/L glucose, 0.3 g/L casamino acid, 2 mg/L 2,4-D, 10 mg/L acetosyringone, 4 g/L Gel-rite, pH 5.2). For cocultivation, this was incubated in the dark at 28° C. for three days.

(4) Bacteria Elimination and Selection

After the completion of cocultivation, Agrobacterium was washed away from the seeds using N6D medium containing 500 mg/L carbenicillin. Subsequently, selection of the transformed seeds was performed according to the following conditions.

First selection: seeds were placed on N6D medium containing 2 mg/L 2,4-D, supplemented with carbenicillin (500 mg/L) and hygromycin (25 mg/L) and incubated at 27° C. to 32° C. for seven days.

Second selection: seeds were placed on N6D medium containing 2 mg/L to 4 mg/L 2,4-D, supplemented with carbenicillin (500 mg/L) and hygromycin (25 mg/L) and incubated at 27° C. to 32° C. for another seven days.

(5) Regeneration

Selected transformed seeds were allowed to regenerate under the following conditions.

First regeneration: selected seeds were placed onto the regeneration medium (MS medium (30 g/L sucrose, 30 g/L sorbitol, 2 g/L casamino acid, 2 mg/L kinetin, 0.002 mg/L NAA, 4 g/L Gel-rite, pH 5.8) supplemented with carbenicillin (500 mg/L) and hygromycin (25 mg/L)), and incubated at 27° C. to 32° C. for two weeks.

Second regeneration: incubation was performed at 27° C. to 32° C. for another two weeks using the same regeneration medium as used in the first regeneration.

(6) Potting

Regenerated transformants were transferred onto rooting medium (MS medium that does not contain hormone and is supplemented with hygromycin (25 mg/L)). After the growth of roots was confirmed, the transformants were potted.

### Example 4

### Genomic PCR

In order to prepare rice genomic DNA, initially, green leaves of wild-type rice plant and nCTB- or mCTB-transgenic rice plant were cut into approximately 5 mm sections, two to three pieces were put in a 1.5 mL microtube, and ground by pushing them with the top of tip. 0.4 mL of DNA extraction buffer (0.2 M Tris-HCl (pH7.5), 0.25 M NaCl, 25 mM EDTA, 0.5% (w/v) SDS) was added, vigorously stirred using a vortex, and then allowed to stand at room temperature for one hour. To the aqueous phase obtained after centrifugation, 0.3 mL of isopropanol was added and mixed. Then, the mixture was centrifuged, pellets were collected and washed with 70% (v/v) ethanol. The obtained pellets were dissolved in 0.1 mL of TE solution to obtain a fraction of rice genomic DNA (PCR experimental protocol for plants, Special Issue of Cell Technology, published by Shujunsha). PCR was performed using the obtained fraction of rice genomic DNA as a template, and the band of CTB gene was confirmed as a result of the agarose electrophoresis of PCR products.

In the PCR, a forward primer F1-Fd (sequence number 11) and reverse primer CTB-KDEL-R (sequence number 4) were used as the primer for amplifying mCTB gene, and a forward primer CTB-F-NcoI (sequence number 3) and reverse primer CTB-KDEL-R (sequence number 4) were used as the primer for amplifying nCTB gene.

As a result, as shown in FIG. 2, when genomic DNAs of mCTB-transgenic rice plant (FIG. 2A) and nCTB-transgenic rice plant (FIG. 2B) were used as a template, the band of CTB gene, 330 bps, was confirmed, in contrast, when genomic DNA of wild-type rice plant was used as a template, the band of CTB gene was not confirmed. In FIG. 2, “#” represents sample numbers (same also in the other figures), and “transformation plasmid” in (A) represents the result of the case where pGPTV-35S-HPT-2.3kGluBpro-sig-mCTB was used as a template and “transformation plasmid” in (B) represents the result of the case where pGPTV-35S-HPT-2.3kGluBpro-sig-nCTB was used as a template.

### Example 5

### Northern Blot Analysis

Total RNA was extracted from six ripening seeds of wild-type rice plant and nCTB- or mCTB-transgenic rice plant. To ground seeds, 0.4 mL of phenol/chloroform/isoamylalchol (25:24:1) was added, vigorously stirred, then 0.4 mL of RNA extraction buffer (0.1 M Tris-HCl (pH 9.0), 0.1 M NaCl, 5 mM EDTA, 1% (w/v) SDS) was added and stirred. To the aqueous phase collected by centrifugation, 0.4 mL of phenol/chloroform/isoamylalchol (25:24:1) was newly added and vigorously stirred. The aqueous phase after centrifugation was corrected, 1 mL of 100% (v/v) ethanol was added, and nucleic acids were precipitated. The precipitates after centrifugation were washed with 70% (v/v) ethanol, dried, and the precipitates were dissolved with 0.3 mL of water. Then, 0.1 mL of 8 M lithium chloride was added, and RNA was precipitated. The precipitates after centrifugation were washed with 2 M lithium chloride and 70% (v/v) ethanol, and dried. Finally, the precipitates were dissolved in 50 μL of water to obtain the total RNA fraction of seeds (Tada, Y. et al. (2003) Plant Biotechnology Journal, vol 1, pp 411-422). 10 μg of the obtained RNA was electrophoresed in a 1% (w/v) formaldehyde-containing agarose gel and transferred onto nylon membranes. Then, transcripts of CTB gene were detected using as a probe PCR amplified fragments of the entire region of CTB gene labeled with ³²p.

As a result, as shown in FIG. 3, in the seeds of mCTB-transgenic rice plant (FIG. 3A) and nCTB-transgenic rice plant (FIG. 3B), transcripts of CTB gene were confirmed, but in the case of seed of wild-type rice plant, transcripts of CTB gene were not confirmed. The amount of RNA analyzed in this experiment was confirmed by staining RNA with ethidium bromide to visualize rRNA.

### Example 6

### Protein Analysis (SDS-PAGE, Western Blot)

Fully ripened seeds of wild-type rice plant and nCTB- or mCTB-transgenic rice plant were ground to a fine powder, and extracted at room temperature for 15 minutes using 50 mM Tris-HCl buffer (pH 6.8) containing 4% SDS, 8 M urea, 5% β-mercaptoethanol and 20% glycerol. Then, the extract was centrifuged and the supernatant was analyzed by 15% acrylamide SDS-PAGE. The experiment was performed in duplicate. In one experiment, protein staining was performed using Coomassie brilliant blue, and in the other, after electrical transfer onto PVDF membranes, western blotting or antibody staining was performed using rabbit anti-CTB serum and HRP-conjugated anti-rabbit IgG antibody.

As a result, as shown in FIG. 4, the CTB to be expressed in the seeds of mCTB-transgenic rice plant was detected not only in the antibody staining (FIG. 4B) but also in the protein staining (FIG. 4A). In contrast, the CTB expressed in the seeds of nCTB-transgenic rice plant was not detected either in the antibody staining (FIG. 4B) or in the protein staining (FIG. 4A). Thus, it was confirmed that the expression level of CTB in the seed of mCTB-transgenic rice plant was higher than that in the seed of nCTB-transgenic rice plant.

Further, in order to confirm whether the expressed CTB had a pentamer structure, fully ripened seeds of wild-type rice plant and mCTB-transgenic rice plant were ground to a fine powder and extracted at room temperature for 15 minutes under non-reductive conditions (50 mM Tris-HCl buffer (pH 6.8) containing 0.1% SDS and 20% glycerol). Then, the extract was centrifuged and the supernatant was analyzed by 15% acrylamide SDS-PAGE. The experiment was performed in duplicate. In one experiment, protein staining was performed using Coomassie brilliant blue, and in the other, after electrical transfer onto PVDF membranes, western blotting or antibody staining was performed using rabbit anti-CTB serum and HRP-conjugated anti-rabbit IgG antibody. The results are shown in FIG. 5. Under non-reductive conditions, both in the protein staining (FIG. 5A) and in the antibody staining (FIG. 5B), most of the bands were observed around 55 kDa to 65 kDa. Thus, it is expected that the CTB expressed in the rice seed has a pentamer structure. The amino acid sequence was analyzed using a protein sequencer (available from Applied Biosystems), by which it was confirmed that of the two bands (10 kDa, 12 kDa) observed as a monomer, the band with lower molecular mass was CTB that comprised a sequence of TPQNI at N-terminus. The amino acid sequence of the band with higher molecular mass could not be analyzed due to possible block at N-terminus, but the band was estimated to be CTB that comprised a glutelin signal sequence.

### Example 7

### Amount of Expressed Protein

For 50 ng to 500 ng of standard CTB derived form bacteria, SDS-PAGE was performed under the conditions similar to those in Example 6 and transferred onto PVDF membranes. Then, antibody staining was performed using rabbit anti-CTB serum and HRP-conjugated anti-rabbit IgG antibody. Thereafter, relative signal intensity was scanned with a scanner (ChemiDoc XRS system: available from Nippon Bio-Rad Laboratories), standard curve of CTB protein was drawn from the data of standard CTB, and the expression level of CTB in the seed of mCTB-transgenic rice plant was determined. The results are shown in FIG. 6. FIG. 6 shows accumulation levels of CTB protein (μg/seed) of three seeds to five seeds each derived from a single transformant line of first filial generation (F1).

### Example 8

### Powderization (Formulation) of mCTB-Transgenic Rice Seed

The chaff of fully ripened rice seed was removed, into the metal corn 2 mL tube of Multi-Beads Shocker (available from Yasui Kikai Corporation), were put five rice seeds after the threshing per tube, set thereto, subjected to vibration three times under the conditions of 2,000 rpm and 7 seconds, and ground to a fine powder. The thus-prepared fine powder of rice seeds was fine such that even in the case where the powder was suspended in 100 mg/mL PBS, the suspension could pass through a disposable feeding needle for mouse (diameter as small as that of 18G needle).

### Example 9

### Oral Immunization by mCTB-Transgenic Rice Seed

100 mg of fully ripened rice seeds, which were ground to a fine powder in Example 8, were suspended in 1 mL of maylon solution (Maylon-P, available from Otsuka Pharmaceutical Co., Ltd.). A group of 6 week- to 8 week-old Balb/c mice (five to six mice) was orally immunized six times every five days by administering the suspension using a disposable feeding needle for mouse (available from Fuchigami-Kikai Ltd.). The mCTB-transgenic rice seed was prepared so that it contained 30 μg of CTB per 100 mg weight by determining the quantity by the method as in Example 7. In the same way, another group as a positive control was orally immunized by administering 30 μg of CTB prepared using E. coli (List Biological Laboratories, USA), and yet another group as a negative control was orally immunized by administering 20 mg of wild-type fully ripened rice seed. Five days after the last immunization, serum, and feces or small intestinal lavage fluid were collected from mice of each group. For feces, extraction was carried out with 1 mL of PBS per 100 mg, and the supernatant after centrifugation was used as a stool sample. Small intestinal lavage was performed as follows. The small intestine was cut away, and intestinal tract was opened and cut into about 2-cm segments. The segments were suspended in 5 mL of PBS and washed. The supernatant after centrifugation was used as a small intestinal lavage fluid.

### Example 10

### Measurement of CTB Specific Antibody in Serum and Feces

The antigen specific antibody was measured according to the method as in Y. Yuki, et al. (Int. Immunol. 4:537-545 (1998)). Specifically, Falcon Microtest III assay plates were coated with 0.5 μg/0.1 mL CTB. Wells were blocked with PBS containing 1% BSA, then serially diluted serum or fecal extract was added, and the plates were incubated at room temperature for 2 hours. After washing the plates with PBS (TPBS) containing 0.01% Tween 20, 0.1 mL of a 1:1000 dilution of peroxidase-conjugated anti-mouse γ, α or μ chain specific antibody (Southern Biotechnology Birmingham, USA) was added to each well and allowed to react at room temperature for 2 hours. After washing with TPBS, colorimetric substrate (3,3′,5,5′-tetramethybenzidine, Moss, Pasadena, USA) was added and developed at room temperature for 10 minutes. Reactions were terminated by addition of 50 μL of 0.5 N hydrochloric acid. For IgG subclass determination, peroxidase bound on antibody to mouse IgG1, IgG2a, IgG2b or IgG3 (Southern Biotechnology Birmingham, USA) was used. Endpoint antigen specific antibody titers were evaluated as the base 2 logarithm (Log₂ value) of the last dilution which gave a reading of OD 450 nm of 0.1 higher than controls.

As a result, as shown in FIG. 7, in the serum obtained from the mice to which mCTB transgenic rice seed powder, or bacteria-derived CTB was orally administered, CTB specific IgG was present that had significantly high antibody titer compared to the serum obtained from the mice to which wild-type rice seed powder was orally administered. In addition, in the serum obtained from the mice to which mCTB transgenic rice seed powder or bacteria-derived CTB was orally administered, CTB specific IgG was present that had the same level of antibody titer.

Next, CTB specific IgG subclass was examined. As a result, as shown in FIG. 8, in the serum obtained from the mice to which mCTB-transgenic rice seed powder or bacteria-derived CTB was orally administered, CTB specific IgG subclass antibodies that had significantly high antibody titers compared to the serum obtained from the mice to which wild-type rice seed powder was orally administered were present, wherein IgG1 had the highest antibody titer, followed by IgG2b and IgG2a. There were no differences in antibody titer of each subclass between the serum obtained from the mice to which mCTB-transgenic rice seed powder and the serum obtained from the mice to which bacteria-derived CTB was orally administered. IgG subclass analysis indicates that the CTB expressed in rice seed can induce Th2 type immune response by oral immunization to mice as bacteria-derived CTB can.

Next, the presence or absence of CTB specific IgA in fecal extract was examined. As a result, as shown in FIG. 9, in the group to which mCTB-transgenic rice seed powder was administered or the group to which bacteria-derived CTB was administered, CTB specific IgA was present that had significantly high antibody titer compared to the group to which wild-type rice seed powder was administered.

Thus, it was confirmed that by orally administering mCTB-transgenic rice seed, not only systemic but also mucosal antigen-specific immune response was induced.

### Example 11

### Demonstration of Presence of Neutralizing Antibody

By utilizing that cholera toxin (CT) causes diarrhea symptom in mice, whether or not the antibody to CTB in serum and secretory fluid, which was induced by oral administration of mCTB-transgenic rice seed, inhibits toxic activity of CT was examined (S. Yamamoto et al. J. Exp. Med 185:1203-1210 (1997)).

The small intestine was removed from non-treated mouse, and ligated with suture to form a 5-cm loop. 1 μg of CT (List Biological Laboratories, USA) was mixed with the serum or small intestinal lavage fluid obtained in Example 9, and injected into the small intestinal loop. The mice were allowed to survive about for 12 hours. Thereafter, the small intestinal loop was removed, cut about every 1 cm, and centrifuged. The water from the body, characteristics of diarrhea symptom, was collected as a supernatant after centrifugation and the amount of the water was measured.

As a result, as shown in FIG. 10, it was confirmed that the antibodies, induced in the serum and small intestinal secretory fluid by oral administration of mCTB-transgenic rice seed or bacteria-derived purified CTB, contained a neutralizing antibody that inhibits the activity of CT. This neutralizing activity was not observed in the serum and small intestinal secretory fluid of the mice to which wild-type rice seed was orally administered.

### Example 12

### Measurement of Neutralizing Antibody Titer

Titers of induced neutralizing antibody were determined by utilizing obstacle effect of CT in CHO cells (S. Yamamoto et al. J. Exp. Med 185:1203-1210 (1997)). Specifically, to a serially diluted serum or small intestinal lavage fluid was added CT (0.01 μg/mL) and mixed. 50 μL of CHO cells (4×10⁵/mL) were added to 50 μL of the mixture and cultivated in DMEM medium containing 5% FCS at 37° C. in 5% CO₂ for 24 hours. Thereafter, living CHO cells were measured. Neutralizing antibody titer was defined as maximum dilution of the serum or small intestinal lavage fluid that has effect to inhibit cell-dysfunction completely.

As a result, as shown in Table 2, the titer of neutralizing antibody, induced in the serum and small intestinal lavage fluid as a result of oral administration of mCTB-transgenic rice seed, was by no means inferior to the titer of neutralizing antibody in the serum and small intestinal lavage fluid induced as a result of oral administration of bacteria-derived purified CTB. In the serum and small intestinal lavage fluid from the mice to which wild-type rice seed was orally administered, neutralizing antibody titer was not confirmed.

TABLE 2

| | Antibody titer | |
|---|---|---|
| | | Small intestinal |
| Sample | Serum | lavage fluid |
| mCTB-transgenic rice seed | 1/1,500 ± 580 | ¼ |
| administered group | | |
| Bacteria-derived purified CTB | 1/3,300 ± 1200 | ⅙ ± 2.3 |
| administered group | | |

Thus, it was confirmed that the mCTB-transgenic rice seed exhibited almost similar immunogenicity to that of bacteria-derived purified CTB when mCTB-transgenic rice seed was used for oral immunization without purifying CTB from mCTB-transgenic rice seed, and it was demonstrated that the mCTB-transgenic rice seed could induce almost the same level of neutralizing antibody titer as that of bacteria-derived purified CTB not only systemically but also mucosally. Therefore, it was confirmed that mCTB-transgenic rice seed could be used as an “edible vaccine”.

### Example 13

### Presence or Absence of Antibody to Rice Seed Protein

In order to demonstrate that antibodies to other proteins contained in the rice seed than CTB were not induced in the serum orally immunized in the way as in Example 9, fully ripened seeds of wild-type rice plant, and nCTB- or mCTB-transgenic rice plant were ground to a fine powder, extracted at room temperature for 15 minutes using 50 mM Tris-HCl buffer (pH 6.8) containing 4% SDS, 8 M urea, 5% β-mercaptoethanol and 20% glycerol. The extract was centrifuged and the supernatant was subjected to 15% acrylamide SDS-PAGE, and then transferred onto PVDF membranes electrically. Antibody staining was performed using the serum of mice obtained in Example 9, i.e., serum of mice which was orally immunized by administering fully ripened seeds of wild-type rice plant, fully ripened seeds of mCTB-transgenic rice plant, or bacteria-derived standard CTB.

As a result, as shown in FIG. 11, in the serum of mice which was orally immunized by administering mCTB-transgenic rice seed or bacteria-derived standard CTB, the presence of antibody to CTB was confirmed, however, the presence of antibodies to other proteins contained in the rice seed than CTB were not confirmed. This indicates that oral immunization of mCTB-transgenic rice seeds induces immune response to CTB, but does not induce immune response to other proteins contained in the rice seed than CTB.

### Example 14

### Generation of Transgenic Rice Plants Producing Chimeric Protein in Which Neutralizing Epitope V3J1 of AIDS Virus is Bound to the C Terminus of CTB

V3J1-KDEL gene, in which DNA (sequence number 1) encoding KDEL (sequence number 2) was added to the 3′ terminus of DNA (sequence number 19) encoding neutralizing epitope V3J1 of AIDS virus, was obtained by chemical synthesis.

In a similar way as in Example 1, V3J1-KDEL gene was subcloned by inserting between the DNA (sequence number 15) encoding a signal peptide of rice glutelin GluB-1 (sequence number 16) and mCTB gene linked downstream thereof; and the terminator of rice glutelin GluB-1 gene (0.6 kbs) (sequence number 18). The DNA (sequence number 15) was linked downstream of the promoter of rice glutelin GluB-1 gene (2.3 kbs) (sequence number 17), and in the subcloning, restriction enzymes, BamHI and SacI were used.

In order to link V3J1-KDEL gene downstream of mCTB gene, the sequence of V3J1-KDEL gene was designed such that DNA (sequence number 20) encoding hinge peptide sequence Gly-Pro-Gly-Pro and restriction enzyme site (ggatcc) was linked downstream of mCTB gene.

In this way, a DNA construct was prepared that contains the promoter of rice glutelin GluB-1 gene, the DNA that is linked downstream thereof and encodes a signal peptide of rice glutelin GluB-1, mCTB gene linked downstream thereof, V3J1-KDEL gene linked downstream thereof, and the terminator of rice glutelin GluB-1 gene, which terminator is linked downstream thereof. This DNA construct was introduced as a HindIII/EcoRI fragment into pGTV-35S-HPT containing hygromycin B resistance gene.

As mentioned above, T-DNA vector pGTV-35S-HPT-2.3kGluBpro-sig-mCTB-V3J1 was constructed in which mCTB gene and V3J1-KDEL gene linked downstream thereof are incorporated.

Thereafter, T-DNA vector was introduced into Agrobacterium in the same way as in Example 2. Then, in the same way as in Example 3, transgenic rice plants were generated that produce chimeric protein (mCTB-V3J1) in which neutralizing epitope V3J1 of AIDS virus is bound to the C terminus of CTB.

### Example 15

### Expression of Chimeric Protein (SDS-PAGE, Western Blotting)

Fully ripened seeds of wild-type rice plant and mCTB-V3J1-transgenic rice plant were ground to a fine powder, extracted at room temperature for 15 minutes using 50 mM Tris-HCl buffer (pH 6.8) containing 4% SDS, 8 M urea, 5% β-mercaptoethanol and 20% glycerol. The extract was centrifuged, and the supernatant was subjected to 15% acrylamide SDS-PAGE and then transferred onto PVDF membranes electrically to perform western blotting or antibody staining using rabbit anti-V3J1 serum and HRP-conjugated anti-rabbit IgG antibody.

As a result, as shown in FIG. 12, CTB-V3J1 protein expressed in the seed of mCTB-V3J1-transgenic rice plant was detected by antibody staining. In the figure, “C” represents the results of wild-type rice plant, and “No. 5”, “No. 26”, and “No. 28” represent the results of mCTB-V3J1-transgenic rice plant.

### Example 16 Southern Blot Analysis

In the same way as in Example 4, genomic DNA was isolated from green leaves of wild-type rice plant, nCTB-transgenic rice plant, and mCTB-transgenic rice plant. Then, 10 μg of DNA was digested with restriction enzyme SacI, subjected to 0.7% agarose gel electrophoresis, and blotted onto Hybond-N+ membranes. Detection of nCTB or mCTB gene was carried out using as a probe double strand DNA encoding nCTB or mCTB, labeled with [α-³²P]dCTP.

As a result, as shown in FIG. 13, in the nCTB-transgenic rice plant (lane 2 in FIG. 13A) and mCTB-transgenic rice plant (lanes 1 to 3 in FIG. 13B), two bands were confirmed, revealing that at least two copies of nCTB or mCTB gene has been introduced in each genome. In the figure, W represents the result of wild-type rice plant.

### Example 17

### Stability of mCTB-Transgenic Rice Seeds

Among seeds of mCTB-transgenic rice plant quantified in Example 7, six lines were stored at room temperature for six months, at room temperature for twelve months, or at 4° C. for twelve months. Then, CTB content was determined and compared with the CTB content of rice seed immediately after the selection of individual. CTB content was determined in the same way as in Example 7, and five rice seeds were used for determination with respect to each storage condition.

As a result, as shown in Table 3, significant difference was not observed between the CTB content of rice seed immediately after the selection of individual and the CTB content of rice seed stored for twelve months at room temperature or 4° C. This result demonstrates that CTB is stable at room temperature for at least twelve months in the rice seed expressing thereof. This means that rice expressing a vaccine gene is much more advantageous than conventional low temperature storage vaccine in terms of storage and transport and that the rice expressing a vaccine gene is vaccine that can be transported without cold chain, which is required for the next generation vaccine.

TABLE 3

| | CTB content (μg) |
|---|---|
| Storage condition | per one rice seed |
| 0 month | 29 ± 4 |
| (immediately after selection) | |
| Six months at room | 29 ± 2 |
| temperature | |
| Twelve months at room | 30 ± 3 |
| temperature | |
| Twelve months at 4° C. | 30 ± 4 |

### Example 18

### Resistance of mCTB-Transgenic Rice Seeds to Digestive Enzyme

Among seeds of mCTB-transgenic rice plant quantified in Example 7, six lines were examined for the resistance to digestive enzyme as follows. 10 mg of mCTB-transgenic rice seed powder was suspended in 0.1 mL of 0.5 mg/mL pepsin (available from Sigma-Aldrich Japan K. K.) dissolved in acetic acid buffer (pH 1.7), and allowed to react at 37° C. for one hour. Then, western blotting analysis was performed in the same way as in Example 6, and CTB, glutelin B1 and prolamin were detected. As a control, 10 mg of wild-type rice seed powder or 15 μg of purified recombinant CTB was treated in the same way. Detections of CTB, glutelin B1 and prolamin were performed using anti-CTB antibody, anti-glutelin B1 antibody and anti-13K prolamin antibody, respectively.

Results are shown in FIG. 14. In the figure, “W”, “T”, and “P” represent wild-type rice seed, mCTB-transgenic rice seed, and purified recombinant CTB, respectively.

As shown in FIG. 14A, in the mCTB-transgenic rice seed after pepsin treatment, CTB remained 75% compared to that in the sample not treated with pepsin, but purified recombinant CTB was completely degraded by pepsin treatment. In addition, as shown in FIG. 14B, 90% of rice storage protein glutelin B1 was degraded by pepsin treatment, however, as shown in FIG. 14C, rice storage protein 13K prolamin was not degraded at all by pepsin treatment.

The above-mentioned results elucidate that expression of vaccine gene in rice dramatically increases resistance of expressed vaccine antigen to pepsin. This means that when rice expressing vaccine antigen is orally administered, the vaccine antigen is degraded in the stomach as less as possible, and thus can reach intestine in an intact state, indicating that the rice expressing vaccine antigen can be used as an oral vaccine.

### Example 19

### Amount of CTB Needed to Induce Antigen Specific Antibody

Since the CTB expressed in rice seed shows resistance to pepsin, it is considered that in order to induce an antigen specific antibody in vivo, smaller amount of CTB is needed when CTB is expressed in the rice seed than the amount of purified recombinant CTB. To demonstrate this, mCTB-transgenic rice seed and purified recombinant CTB were dissolved or suspended in water so that the amount of CTB orally immunized to mice was 30 μg, 10 μg, 3 μg, 1 μg, 0.3 μg, or 0.1 μg. Mice were orally immunized three times every one week, and one week after the last immunization, antibody titers in serum and feces were measured.

As a result, as shown in FIG. 15, it was revealed that mCTB-transgenic rice seed can induce the same level of antigen-specific immune response (serum anti-CTB IgG (systemic immune response, FIG. 15A) and feces anti-CTB IgA (mucosal immune response, FIG. 15B)) as that of purified recombinant CTB at a dosage (in terms of CTB amount) about one third to about one tenth of that of purified recombinant CTB, indicating that the CTB expressed in rice can induce antigen specific immunity at lower concentration by escaping degradation in digestive organ. This effect was especially remarkable in mucosal immune response, FIG. 15B).

### Example 20

### Immune Electron Microscopy of mCTB-Transgenic Rice Seeds

In order to examine localization of CTB in the endosperm cell of mCTB-transgenic rice seed, rice seeds on the twelfth day after flowering were fixed with 4% paraformaldehyde, dehydrated, and then embedded in LR-white resin to prepare ultrathin sections on Ni grids. These sections were treated with rabbit anti-mCTB antibody (1:500 dilution), subjected to blocking, allowed to react with gold-particle conjugated goat anti-rabbit IgG, then washed, stained with 2% uranyl acetate, and observed with a transmission electron microscope.

Results are shown in FIG. 16. In the figure, A is an observed picture of mCTB-transgenic rice seed, and B is an observed picture of wild-type rice seed. In the figure, large white part, light black part (arrow), light grey part (arrow), and small black dot represent starch granule, type II storage protein body containing storage protein glutelin, type I storage protein body containing storage protein prolamin, and CTB, respectively.

As shown in FIG. 16A, CTB was observed in type I storage protein body (protein body I) and type II storage protein body (protein body II), and in the spaces therebetween (cytoplasm, endoplasmic reticula, etc.) In contrast, as shown in FIG. 16B, in the case of wild-type rice seed, significant staining of CTB was not observed. To consider these observations together with the results of Example 18 (90% of rice storage protein glutelin B1 was degraded by pepsin treatment, however, rice storage protein 13K prolamin was not degraded at all by pepsin treatment), it indicates that the resistance of CTB expressed in rice to pepsin is provided by the presence of CTB in the place other than type II storage protein body, particularly in the type I storage protein body.

### Example 21

### Uptake of CTB into Mucosal Inductive Tissue

In order to demonstrate that orally administered mCTB-transgenic rice seeds escape degradation in digestive organ, reaches intestinal tract, and is taken up by cells (particularly, M cells) that are present in e.g. a Peyer's patch which is an mucosal inductive tissue and that are capable of taking up antigen, intestinal tract of non-treated was litigated, into which suspension of mCTB-transgenic rice seed or wild-type rice seed powder was added. After thirty minutes, Peyer's patch was removed and washed. Then, paraffin sections were prepared and HRP (horseradish peroxidase) enzyme staining was performed using anti-CTB antibody (IgG fraction) and Ulex Europeus Agglutinin (UEA)-1 lectin. M cells were stained with lectin UEA-1 that recognizes fucose, and goblet cells that secrete mucus present in the epithelial cell layer, and Paneth cells are also stained with lectin UEA-1 that are present at so-called crypt.

Results are shown in FIG. 17. In FIG. 17, three pictures in column indicated as “mCTB-transgenic rice seed UEA-1” represent the results of UEA-1 staining when mCTB-transgenic rice seed was administered. Three pictures in column indicated as “wild-type rice seed UEA-1” represent the results of UEA-1 staining when wild-type rice seed was administered. Three pictures in column indicated as “mCTB-transgenic rice seed anti-CTB antibody” represent the results of anti-CTB antibody staining when mCTB-transgenic rice seed was administered. Three pictures in column indicated as “wild-type rice seed anti-CTB antibody” represent the results of anti-CTB antibody staining when wild-type rice seed was administered. In addition, four pictures in row indicated as A represent the results of staining of specialized epithelium of Peyer's patch domes (arrows in A represent M cells). Four pictures in row indicated as B represent the results of staining of villous layer of epithelial cells (* in B represent goblet cells). Four pictures in row indicated as C represent the results of staining of crypt tissue (in C, arrows represent Paneth cells, and * in B represents goblet cells).

As shown in FIG. 17, in any cases of administrations of mCTB-transgenic rice seed and wild-type rice seed, specialized epithelium of Peyer's patch domes (A), villous layer of epithelial cells (B) and crypt tissue (C) were stained with UEA-1 staining. GM-1 ganglioside, a receptor specific to CTB, is present on any epithelial cells. Thus, when the same location was stained by mirror staining using an anti-CTB antibody, the presence of CTB was confirmed in the specialized epithelium of Peyer's patch domes (A), villous layer of epithelial cells (B), and crypt tissue (C), and especially, abundant CTB was confirmed in M cells, stained with UEA-1 lectin, of specialized epithelium of Peyer's patch domes (A). This indicates that the CTB expressed in rice escapes digestion by enzyme in the stomach, reaches small intestine, is taken up by M cells present in Peyer's patch which is mucosal inductive tissue, and is presented as an antigen by antigen-presenting cells present immediately beneath the M cells. Thus, it was revealed that when vaccine antigen expressed in rice is orally administered, the vaccine antigen escapes degradation in the stomach, reaches intestinal tract, and is efficiently taken up by M cells that are present in e.g. a Peyer's patch.

### Example 22

### Generation of Transgenic Rice Plants with Botulinum Toxin A-neutralizing Domain (HcA, Molecular Mass 50 KDa) Gene

A DNA fragment was chemically synthesized (sequence number 21) in which restriction enzyme NcoI recognition sequence and; KDEL sequence and restriction enzyme SacI recognition sequence are added to 5′ terminus and 3′ terminus of plant codon optimized form of HcA (PlaHcA) gene, respectively (hereinafter, referred to as “PlaHcA-KDEL gene”. The codon of PlaHcA-KDEL gene was optimized for the expression in rice as in Example 1. Amino acid sequence of protein encoded by PlaHcA-KDEL gene is shown in sequence number 22.

In the same way as in Example 1, PlaHcA-KDEL gene was subcloned by inserting between the DNA (sequence number 15) encoding a signal peptide of rice glutelin GluB-1 (sequence number 16); and the terminator of rice glutelin GluB-1 gene (sequence number 18). The DNA (sequence number 15) was linked downstream of the promoter of rice glutelin GluB-1 gene (sequence number 17), and in the subcloning, restriction enzymes, NcoI and SacI were used. In this way, a DNA construct was prepared that contains the promoter of rice glutelin GluB-1 gene, the DNA that is linked downstream thereof and encodes a signal peptide of rice glutelin GluB-1, PlaHcA gene linked downstream thereof, the DNA that is linked downstream thereof and encodes KDEL sequence, and the terminator of rice glutelin GluB-1 gene, which terminator is linked downstream thereof. This DNA construct was introduced as Sse8387I/EcoRI fragment into pTL7 (International Publication No. WO 2004/087910) to construct pTLGluB1-PlaHcA. Further, a Sse83871 fragment, in which (1) hygromycin B resistance gene (HPT), (2) recombinase gene (R) of a yeast R/RS site-specific recombinant system, and (3) gene for cytokinin synthetic enzyme (ipt) and terminator of the gene for cytokinin synthetic enzyme (ipt terminator) linked downstream thereof are sandwiched between two recombination sequences (RS) in the same orientation of a yeast R/RS site-specific recombinant system, was introduced into pTLGluB1-PlaHcA, wherein the HPT is linked between a promoter of cauliflower mosaic virus 35S (35S promoter) and terminator of nopaline synthase (Nos terminator), the recombinase gene (R) is linked between a promoter of cauliflower mosaic virus 35S and terminator of nopaline synthase, and the ipt is linked downstream of a promoter of cauliflower mosaic virus 35S and is derived from Agrobacterium T-DNA. As mentioned above, a T-DNA vector pTLGluB1-PlaHcA-130HmintrepR into which PlaHcA gene is incorporated (see FIG. 18) was constructed. This structure is MAT vector (registered mark) that allows the removal of drug resistance gene (International Publication No. WO 2004/087910).

Thereafter, in the same way as in Example 2, T-DNA vector was introduced into Agrobacterium, and then transgenic rice plants that produce an Hc domain of botulinum toxin (HcA) were generated according to the method described in Example 1 of WO 2004/087910.

### Example 23

### Expression and Expression Site of HcA Protein of Transgenic Rice Plants with Botulinum Toxin A-neutralizing Domain Gene (PlaHcA Gene)

In the same way as in Example 6, fully ripened seeds of wild-type rice plant and PlaHcA-transgenic rice plant were ground to a fine powder, extracted at room temperature for 15 minutes using 50 mM Tris-HCl buffer (pH 6.8) containing 4% SDS, 8 M urea, 5% β-mercaptoethanol and 20% glycerol. The extract was centrifuged and the supernatant was subjected to 15% acrylamide SDS-PAGE, and transferred onto PVDF membranes electrically. Then, western blotting or antibody staining was performed using rabbit anti-HcA serum and HRP-conjugated anti-rabbit IgG antibody. As a result, as shown in FIG. 19, HcA band was confirmed around a molecular mass of 50 KDa in 4 lines (lanes 3, 4, 6, 7) of PlaHcA transgenic rice seeds; however, in wild-type (lane 2) and 2 lines (lanes 5, 8), the band was not confirmed at the same position. Since this test was carried out using rice seeds of self pollinated F1, there were rice seeds not expressing HcA such as two lines of lanes 5 and 8. However, it was demonstrated that it is possible to allow PlaHcA-transgenic rice plants to express HcA with a molecular mass of 50KDa. Further, the localization of HcA in rice endosperm cell was observed by the immune electron microscopy as was in Example 20. As in the case of CTB, HcA was observed in type I storage protein body and type II storage protein body, and in the spaces therebetween (cytoplasm, endoplasmic reticula, etc.).

### BRIEF DESCRIPTION OF DRAWINGS

FIG. 1 shows structures of a T-DNA vector pGPTV-35S-HPT-2.3kGluBpro-sig-mCTB into which mCTB gene is incorporated, and a T-DNA vector pGPTV-35S-HPT-2.3kGluBpro-sig-nCTB into which nCTB gene is incorporated.

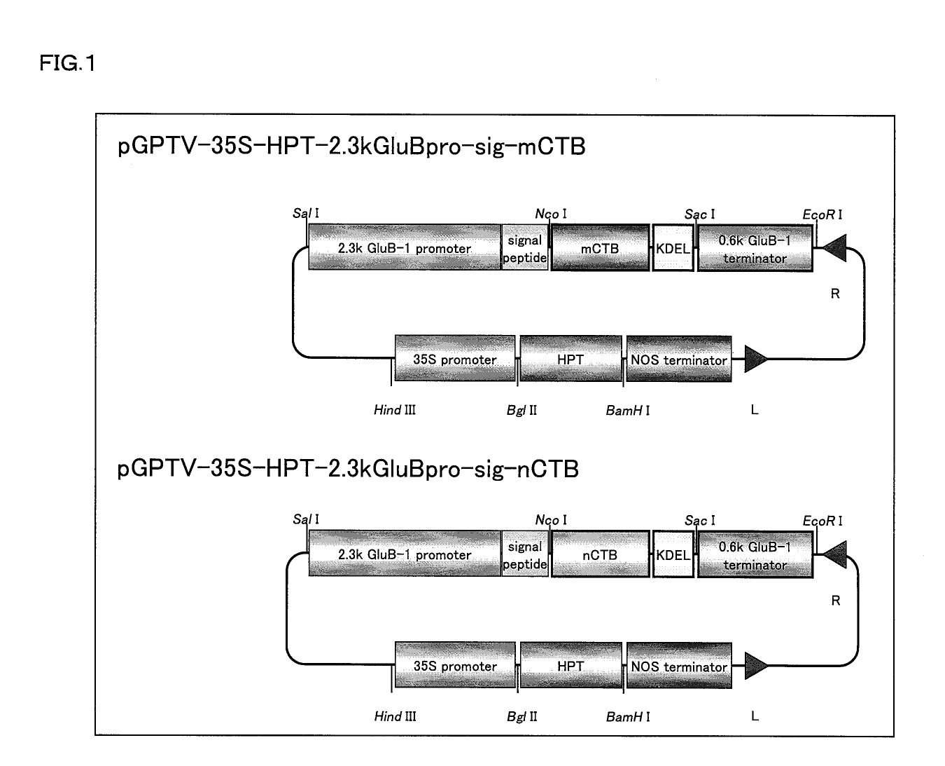

FIG. 2 shows the results of PCR using as a template mCTB-transgenic rice plant (FIG. 2A) and nCTB-transgenic rice plant (FIG. 2B).

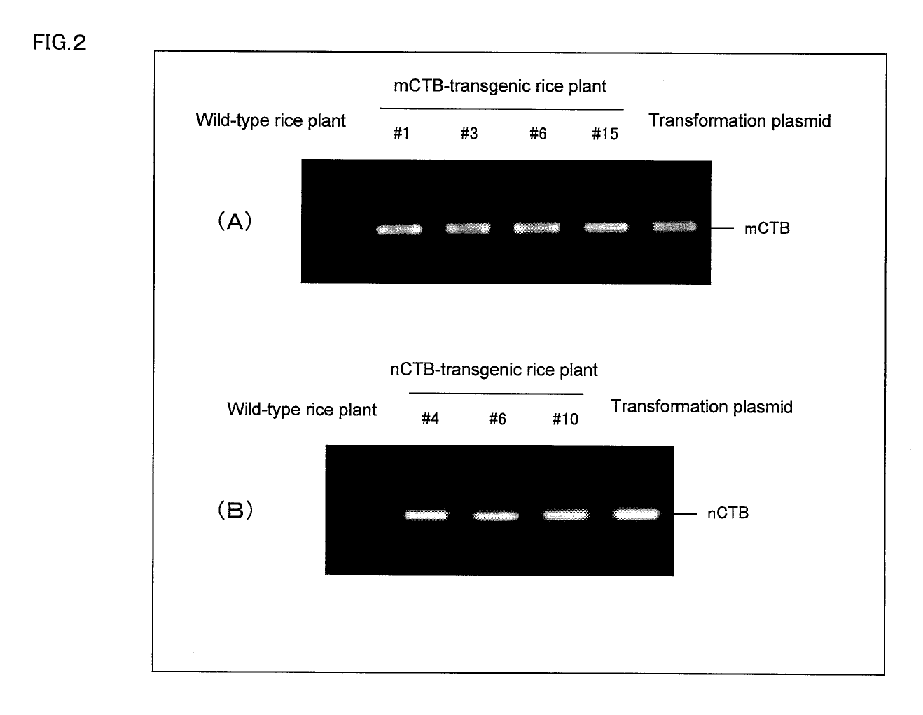

FIG. 3 shows the results of northern blot analysis of seeds of mCTB-transgenic rice plant (FIG. 3A) and nCTB-transgenic rice plant (FIG. 3B).

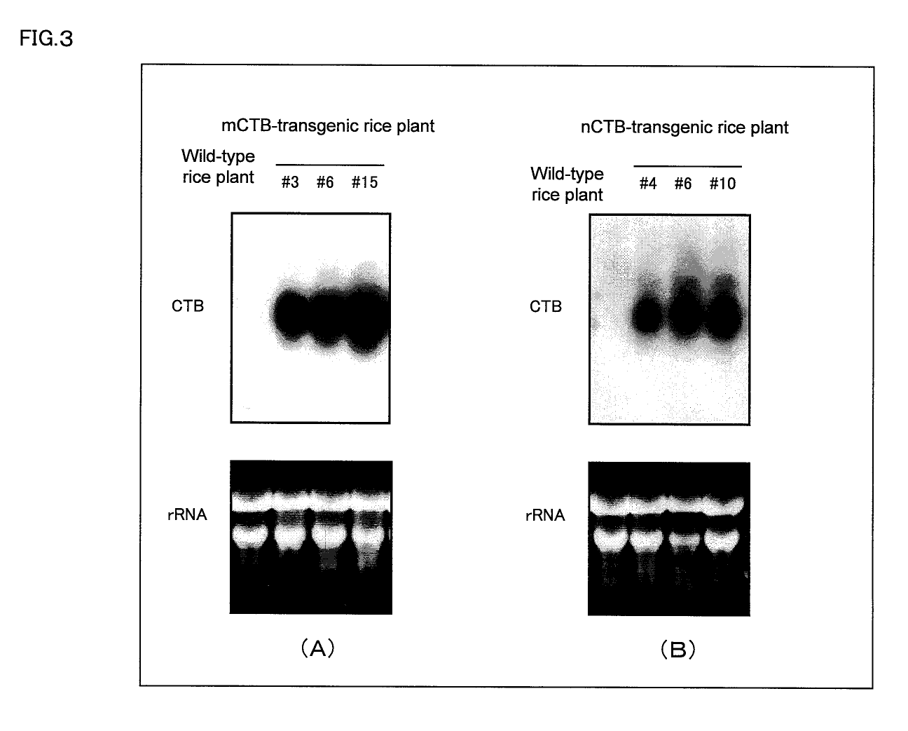

FIG. 4 shows the results of protein (FIG. 4A) and antibody staining (FIG. 4B) for seeds of mCTB-transgenic rice plant and nCTB-transgenic rice plant.

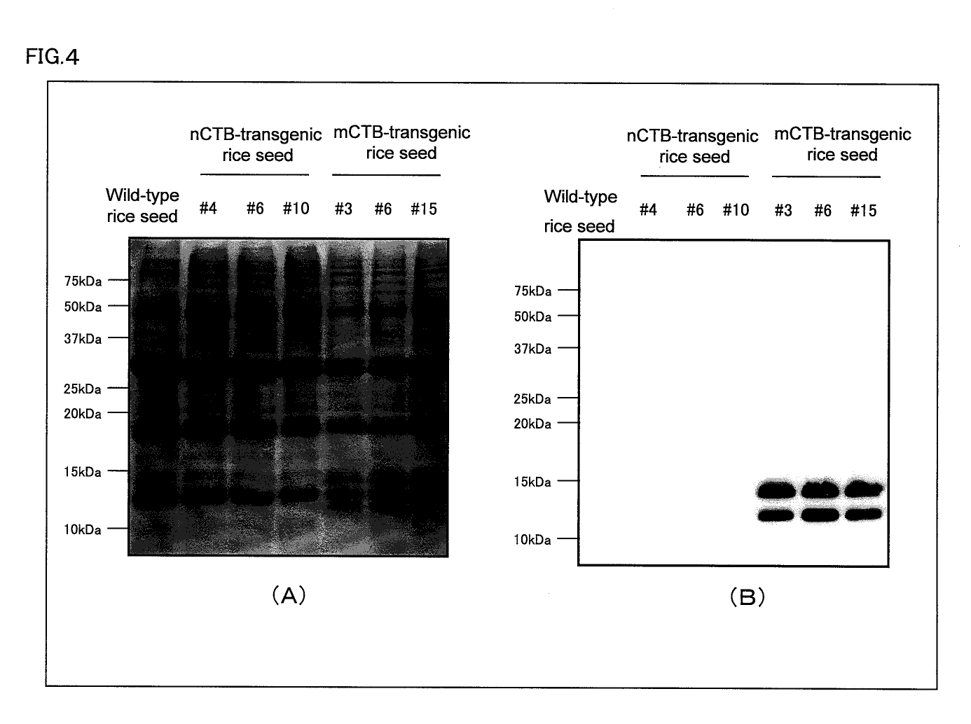

FIG. 5 shows the results of protein staining (FIG. 5A) and antibody staining (FIG. 5B) for seeds of mCTB-transgenic rice plant.

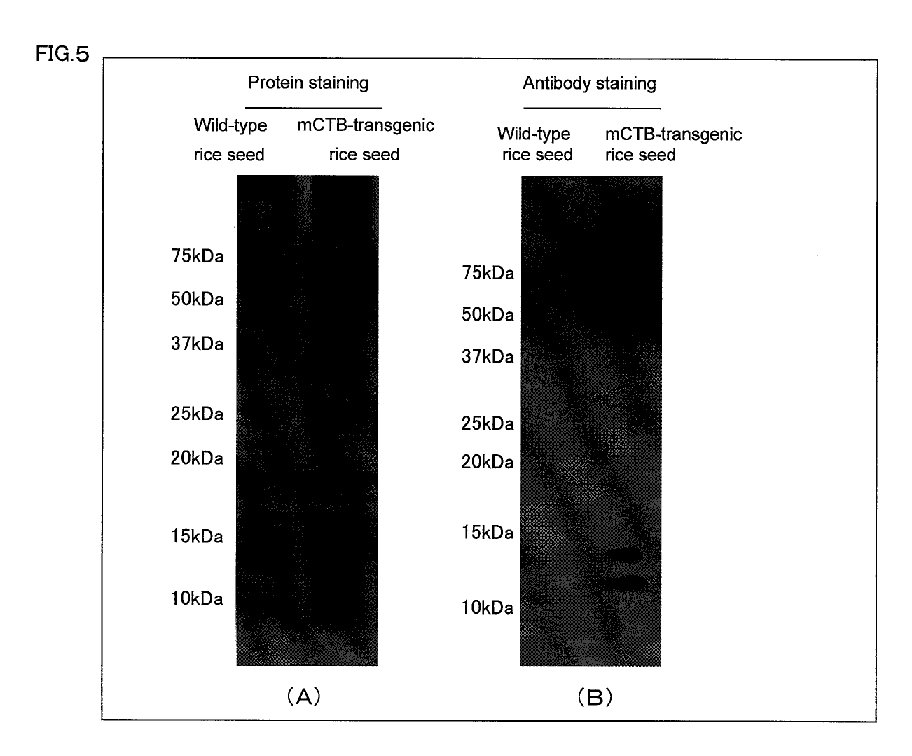

FIG. 6 shows the results of determination of CTB expression level in seeds of mCTB-transgenic rice plant.

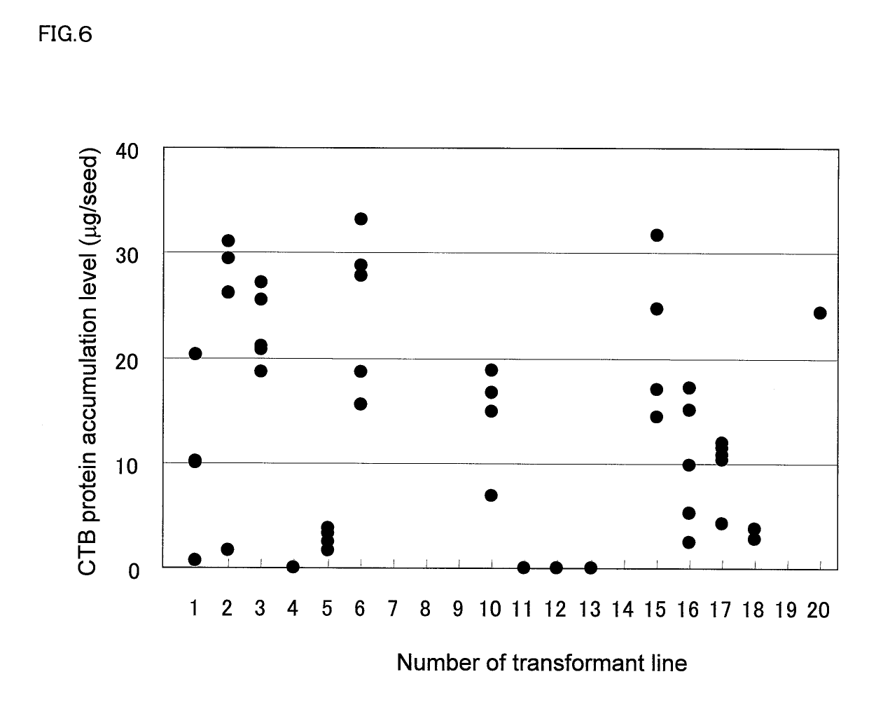

FIG. 7 shows measurement results of antibody titers of serum obtained from the mice to which wild-type rice seed powder, mCTB-transgenic rice seed powder, or bacteria-derived CTB was orally administered.

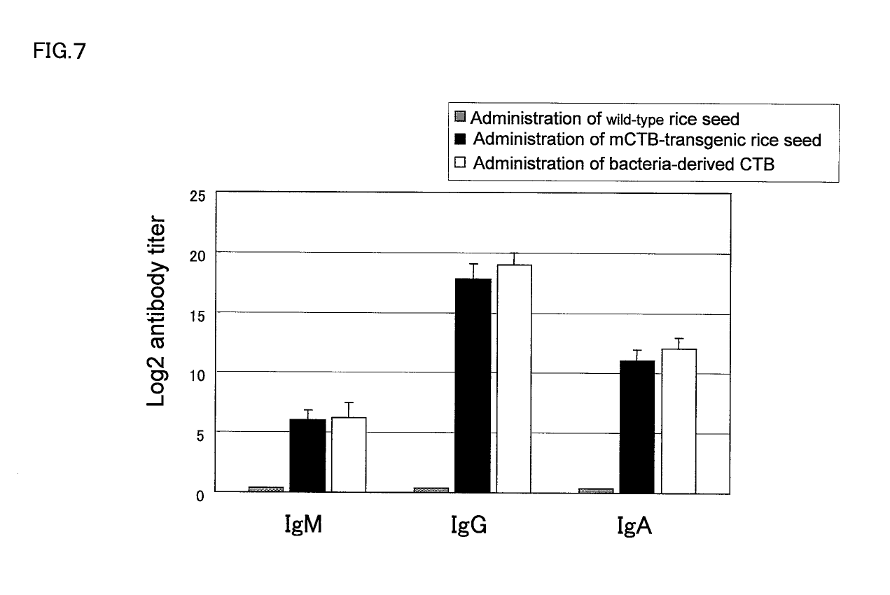

FIG. 8 shows measurement results of antibody titers of CTB specific IgG subclass antibodies contained in the serum obtained from the mice to which wild-type rice seed powder, mCTB-transgenic rice seed powder, or bacteria-derived CTB was orally administered.

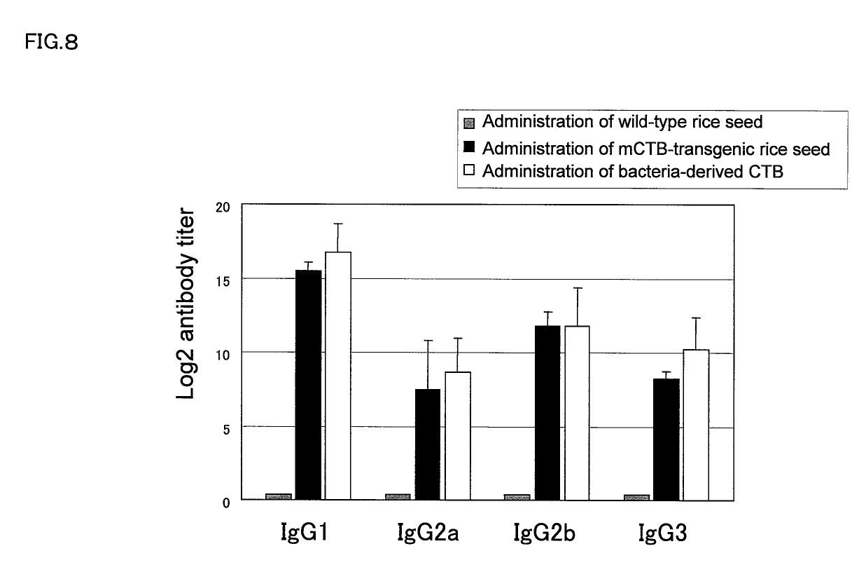

FIG. 9 shows measurement results of antibody titer of CTB specific IgA contained in the fecal extract of the mice to which wild-type rice seed powder, mCTB-transgenic rice seed powder, or bacteria-derived CTB was orally administered.

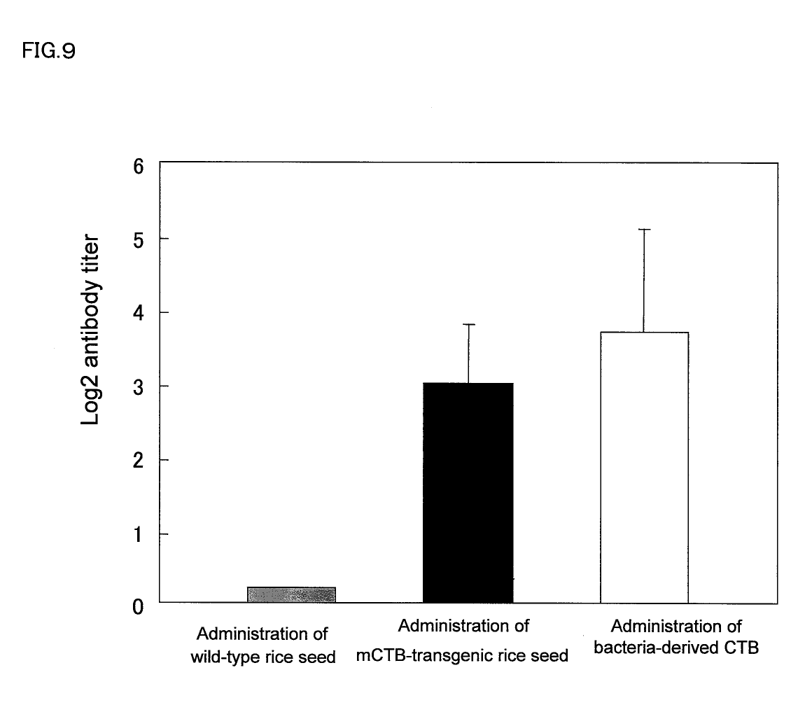

FIG. 10 shows that the antibodies, induced in the serum and small intestinal secretory fluid of the mice to which wild-type rice seed powder, mCTB-transgenic rice seed powder, or bacteria-derived CTB was orally administered, contain a neutralizing antibody which inhibits the activity of CT.

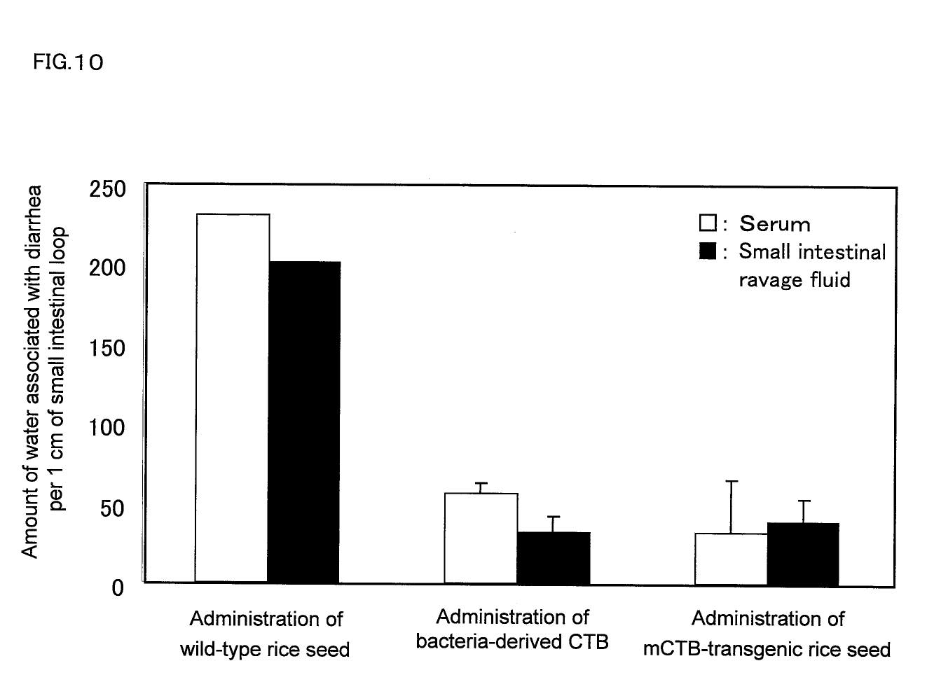

FIG. 11 shows a presence of antibody to CTB in serum of mice to which wild-type rice seed powder, mCTB-transgenic rice seed powder, or bacteria-derived CTB was orally administered and an absence of antibodies to other proteins contained in the rice seed than CTB.

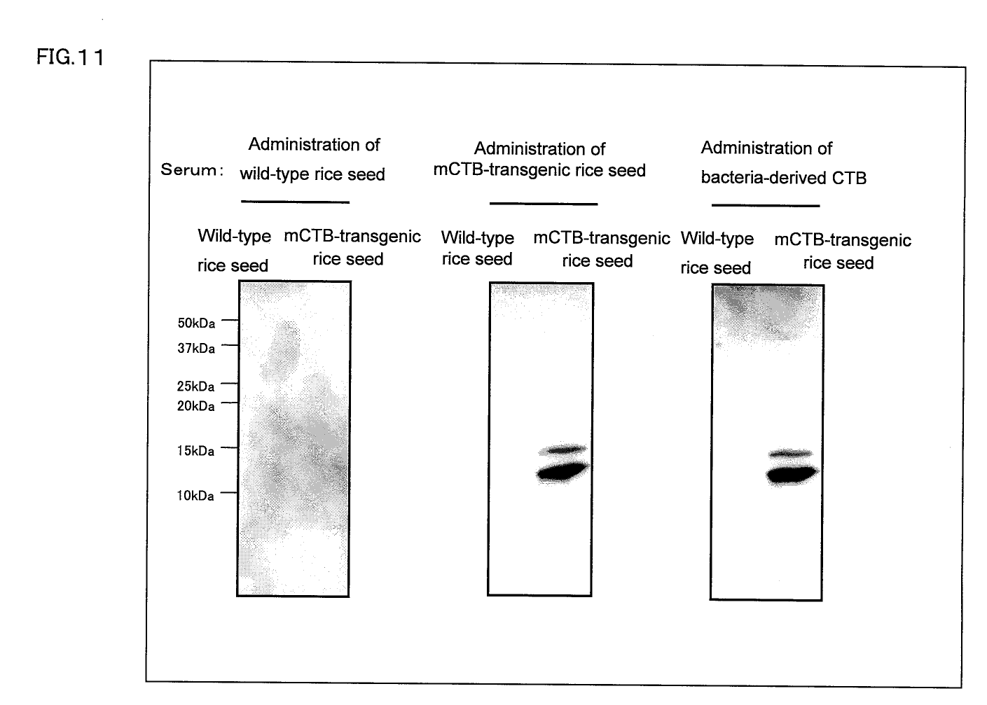

FIG. 12 shows the results of antibody staining for seeds of wild-type rice plant and mCTB-V3J1-transgenic rice plant.

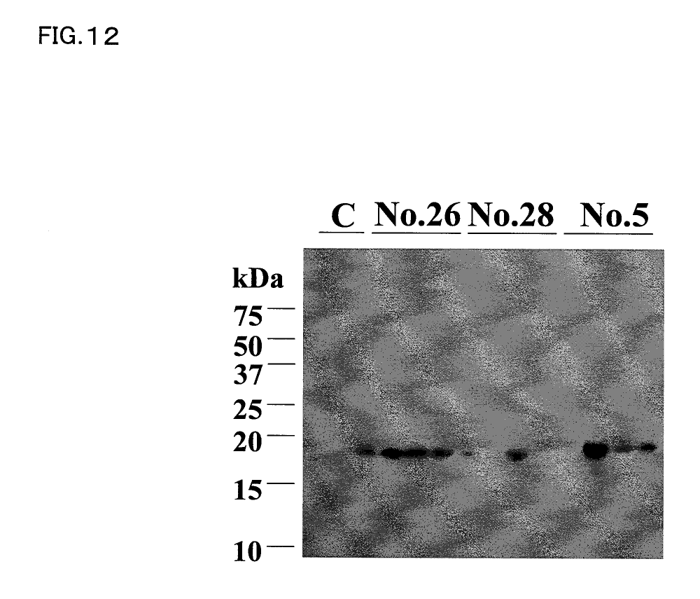

FIG. 13 shows the results of Southern blot analysis of genomic DNA isolated from nCTB-transgenic rice plant (FIG. 13A) and mCTB-transgenic rice plant (FIG. 13B).

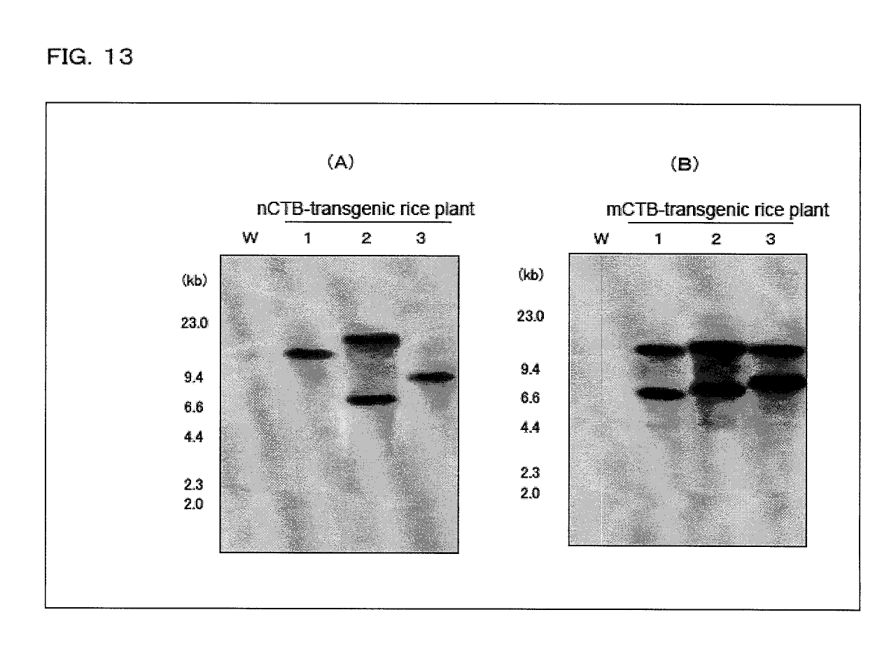

FIG. 14 shows measurement results of resistance of mCTB-transgenic rice seed to digestive enzyme (pepsin).

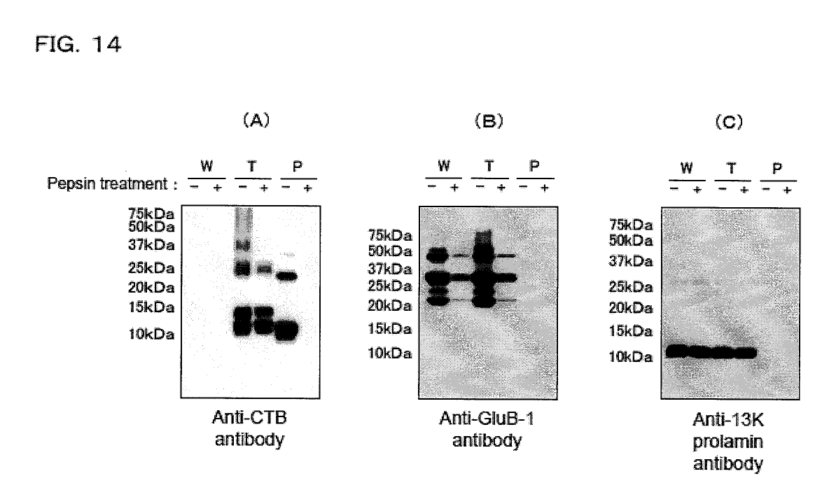

FIG. 15 shows a dosage of CTB needed to induce an antigen-specific immune response (serum anti-CTB IgG (systemic immune response, FIG. 15A) and feces anti-CTB IgA (mucosal immune response, FIG. 15B).

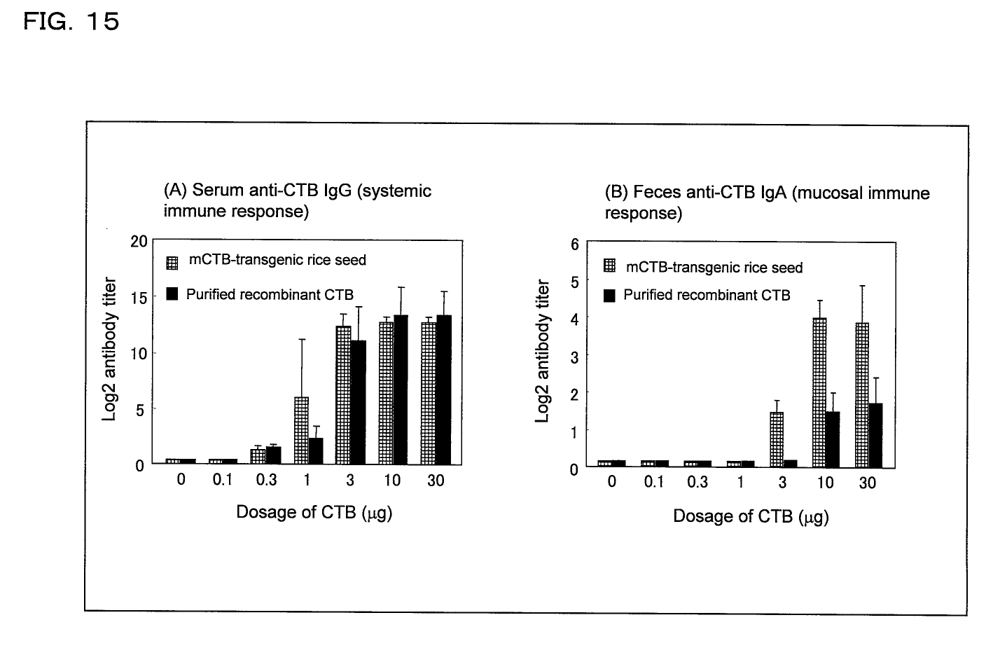

FIG. 16 shows a localization of CTB in the endosperm cells of mCTB-transgenic rice seed (FIG. 16A) and wild-type rice seed (FIG. 16B).

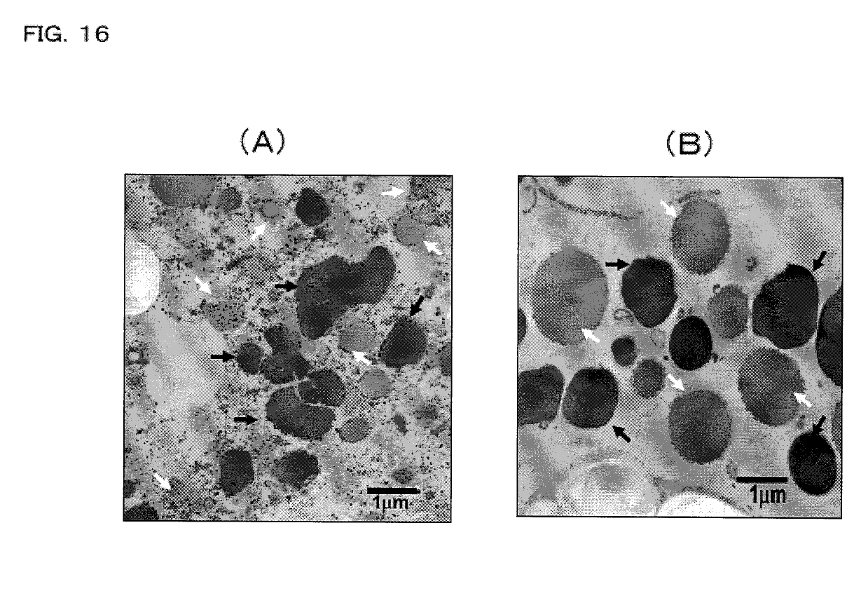

FIG. 17 shows uptake of mCTB into mucosal inductive tissue.

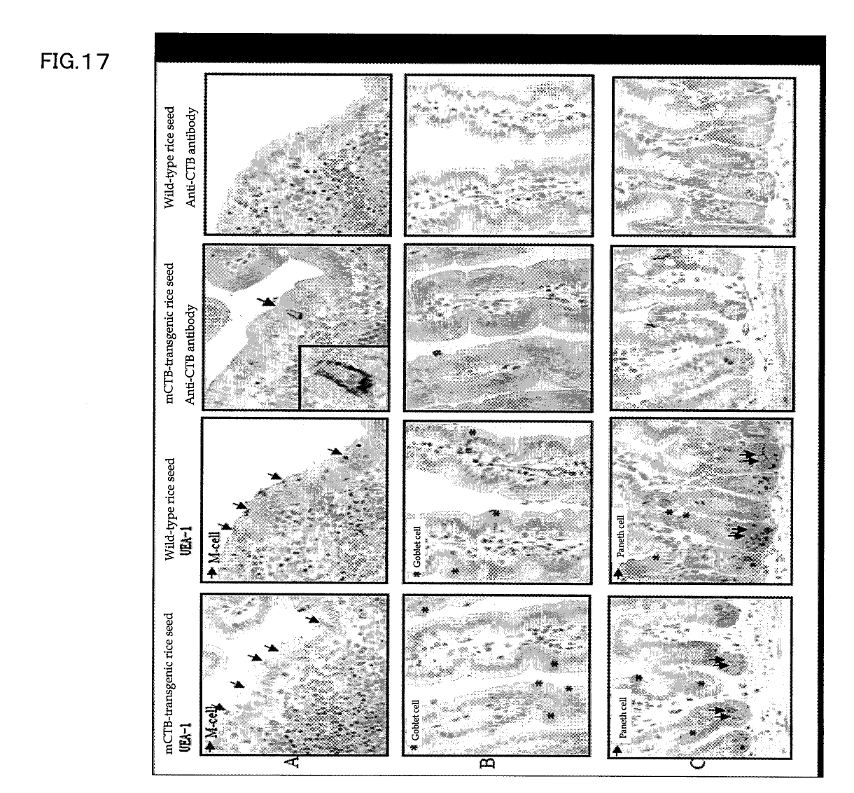

FIG. 18 shows a structure of a T-DNA vector pTLGluB1-PlaHcA-130HmintrepR into which PlaHcA gene is incorporated.

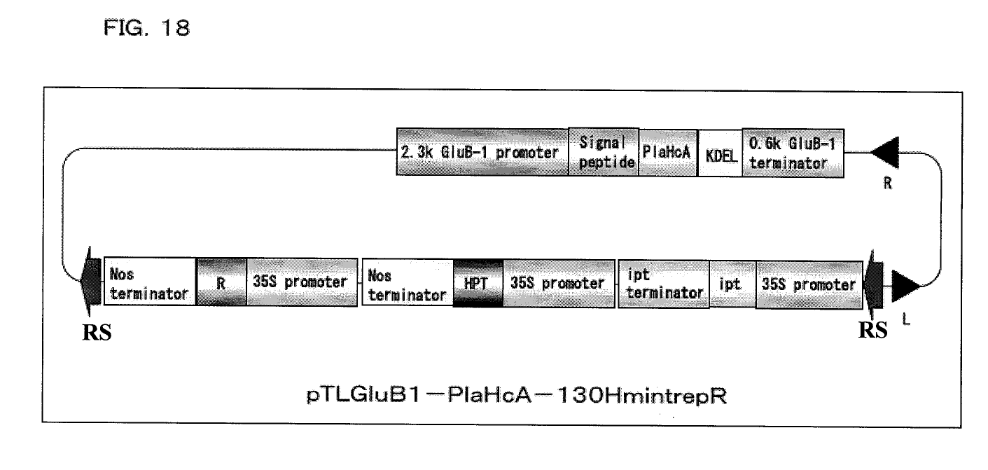

FIG. 19 shows the results of western blotting or antibody staining for seeds of botulinum toxin A-neutralizing domain gene-(PlaHcA gene-) introduced rice.

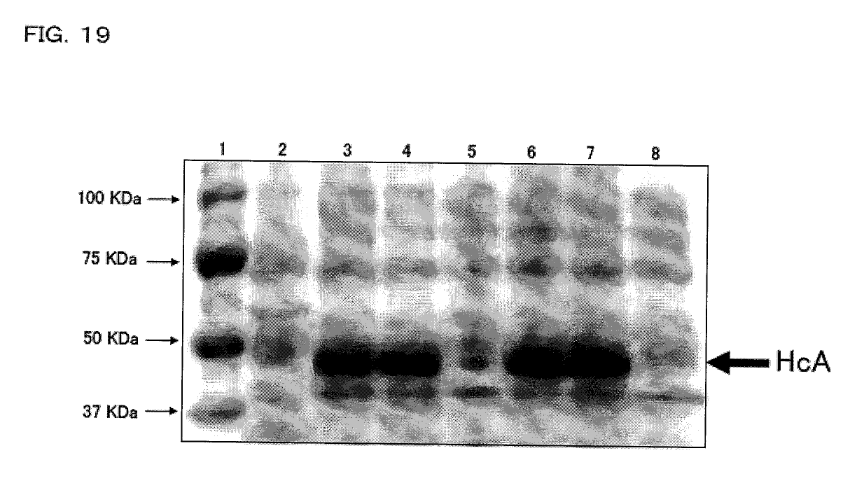

1. A transgenic rice plant comprising a genomic DNA,
wherein a DNA construct is incorporated into the genomic DNA so that the DNA construct is capable of being expressed, wherein the DNA construct comprises a DNA encoding an antigenic protein, and a rice endosperm specific promoter linked upstream thereof.

2. The transgenic rice plant according to claim 1, wherein the antigenic protein as a monomer has a molecular mass of 100,000 dalton or less.

3. The transgenic rice plant according to claim 1, wherein the antigenic protein is a subunit constituting a homomultimer.

4. The transgenic rice plant according to claim 3, wherein the homomultimer has a molecular mass of 1,000,000 dalton or less.

5. The transgenic rice plant according to claim 1, wherein the antigenic protein is a subunit constituting a heteromultimer.

6. The transgenic rice plant according to claim 5, wherein the DNA construct comprises a DNA encoding each subunit constituting the heteromultimer.

7. The transgenic rice plant according to claim 6, wherein the heteromultimer has a molecular mass of 1,000,000 dalton or less.

8. The transgenic rice plant according to claim 1, wherein the antigenic protein is one of a cholera toxin B subunit, a fusion protein of a cholera toxin B subunit and a neutralizing epitope of AIDS virus, and an Hc domain of botulinum toxin.

9. The transgenic rice plant according to claim 1, wherein the rice endosperm specific promoter is a promoter of a gene encoding a storage protein in rice endosperm.

10. The transgenic rice plant according to claim 9, wherein the gene encoding a storage protein in rice endosperm is one of a glutelin gene, a prolamin gene and a globulin gene.

11. The transgenic rice plant according to claim 10, wherein the glutelin gene is glutelin GluB-1 gene.

12. The transgenic rice plant according to claim 1, wherein the DNA construct comprises a rice endosperm specific terminator which is linked downstream of the DNA encoding an antigenic protein.

13. The transgenic rice plant according to claim 12, wherein the rice endosperm specific terminator is alternator of a gene encoding a storage protein in rice endosperm.

14. The transgenic rice plant according to claim 13, wherein the gene encoding a storage protein in rice endosperm is one of a glutelin gene, a prolamin gene and a globulin gene.

15. The transgenic rice plant according to claim 1, wherein the DNA construct comprises a DNA encoding a signal peptide of a storage protein in rice endosperm so that a fusion protein is produced by the expression of the DNA construct, wherein the DNA encoding a signal peptide is linked upstream of the DNA encoding an antigenic protein, wherein the fusion protein comprises the antigenic protein and the signal peptide attached to the N terminus thereof.

16. The transgenic rice plant according to claim 15, wherein the signal peptide of a storage protein in rice endosperm is a signal peptide of one of glutelin, prolamin and globulin.

17. The transgenic rice plant according to claim 1, wherein the DNA construct comprises a DNA encoding an endoplasmic reticulum-localization signal peptide so that a fusion protein is produced by the expression of the DNA construct, wherein the DNA encoding an endoplasmic reticulum-localization signal peptide is linked downstream of the DNA encoding an antigenic protein, wherein the fusion protein comprises the antigenic protein and the endoplasmic reticulum-localization signal peptide attached to the C terminus thereof.

18. The transgenic rice plant according to claim 17, wherein the endoplasmic reticulum-localization signal peptide comprises an amino acid sequence described in sequence number 2.

19. The transgenic rice plant according to claim 1, wherein at least one codon contained in the DNA encoding an antigenic protein is modified to a plant codon.

20-23. (canceled)

24. The transgenic rice plant according to claim 1, wherein oral administration of seed powder of the transgenic rice plant to an animal is capable of inducing systemic and mucosal immune responses specific to the antigenic protein, and the antigenic protein in the seed powder has an increased resistance to pepsin compared to a purified antigenic protein.

25. The transgenic rice plant according to claim 24, wherein the antigenic protein in seed of the transgenic rice plant is stable at room temperature for twelve months or more.

26. The transgenic rice plant according to claim 24, wherein the antigenic protein is taken up by M cells of mucosal inductive tissue by orally administering the seed powder to the animal.

27. A processed rice product, comprising one of rice and an antigenic protein which are obtained from a transgenic rice plant,
wherein the transgenic rice plant comprises a genomic DNA,
wherein a DNA construct is incorporated into the genomic DNA so that the DNA construct is capable of being expressed, wherein the DNA construct comprises a DNA encoding an antigenic protein, and a rice endosperm specific promoter linked upstream thereof.

28. A vaccine composition, comprising as an active ingredient a processed rice product,
wherein the processed rice product comprises one of rice and an antigenic protein which are obtained from a transgenic rice plant,
wherein the transgenic rice plant comprises a genomic DNA,
wherein a DNA construct is incorporated into the genomic DNA so that the DNA construct is capable of being expressed, wherein the DNA construct comprises a DNA encoding an antigenic protein, and a rice endosperm specific promoter linked upstream thereof.

29. A method for inducing an immune response in an animal other than a human, comprising mucosally administering a vaccine composition to the animal other than the human,
wherein the vaccine composition comprises as an active ingredient a processed rice product,
wherein the processed rice product comprises one of rice and an antigenic protein which are obtained from a transgenic rice plant,
wherein the transgenic rice plant comprises a genomic DNA.
wherein a DNA construct is incorporated into the genomic DNA so that the DNA construct is capable of being expressed wherein the DNA construct comprises a DNA encoding an antigenic protein, and a rice endosperm specific promoter linked upstream thereof

30. A method for inducing an immune response in a human, comprising mucosally administering a vaccine composition to the human,
wherein the vaccine composition comprises as an active ingredient a processed rice product,
wherein the processed rice product comprises one of rice and an antigenic protein which are obtained from a transgenic rice plant,
wherein the transgenic rice plant comprises a genomic DNA,
wherein a DNA construct is incorporated into the genomic DNA so that the DNA construct is capable of being expressed, wherein the DNA construct comprises a DNA encoding an antigenic protein, and a rice endosperm specific promoter linked upstream thereof.
